(19) United States
(12) Patent Application Publication
(10) Pub. No.: US 2010/0099112 A1
(43) Pub. Date: Apr. 22, 2010

(54) QUANTITATIVE LATERAL FLOW SYSTEM AND ASSAY

(76) Inventors: Siliang Zhou, Hayward, CA (US); William J. Rutter, San Francisco, CA (US); Ning Liu, Sichuan (CN)

Correspondence Address: GLOBAL PATENT GROUP - RLA, GLOBAL PATENT GROUP, LLC, 10411 Clayton Road, Suite 304, ST. LOUIS, MO, 63131, US

(21) Appl. No.: 11/569,870

(22) PCT Filed: Jun. 2, 2005
(86) PCT No.: PCT/US2005/019348

§ 371 Date: Jan. 4, 2010

Related U.S. Application Data
(60) Provisional application No. 60/576,327, filed on Jun. 2, 2004.
(60) Provisional application No. 60/592,202, filed on Jul. 29, 2004.

(51) Int. Cl. G01N 33/53; C12M 1/34

(52) U.S. Cl. 435/7.1; 435/287.8

(57) ABSTRACT

The present invention relates to a lateral flow assay and system, including a test strip, for detection and quantification of analytes in samples, such as samples containing cells and fluid, where the assay is volume independent, and the sample size is less than about 100 μl, where the test strip includes a first membrane such as a sample filter, that is in capillary contact with an optional second membrane, such as a fluid collector, the second membrane, if present is in capillary contact with an optional third membrane, such as a conjugate pad containing a mobilizable detectable agent, or with a fourth membrane, which is a chromatographic strip, which optionally contains a mobilizable detectable agent, all such membranes being in fluid contact with a fifth membrane, such as a buffer pad, a sixth membrane, such as an absorbent pad, optionally a seventh membrane, which is also an absorbent pad, a capture band for capturing the analyte and at least one control band, or alternatively, the chromatographic strip contains the mobilizable detectable agent in place of a conjugate pad, where the test strip is configured to support removal of red blood cells from the sample and to allow uni-directional or bi-directional fluid flow of fluid from the sample filter to the capture band to be retained therein and detected thereon.

### CROSS-REFERENCE TO RELATED APPLICATIONS

This application hereby claims priority from U.S. Provisional Patent Application Ser. No. 60/576,327, filed Jun. 2, 2004 by Zhou et al. and entitled “Quantitative Lateral Flow System and Assay,” as well as from U.S. Provisional Application Ser. No. 60/592,202, filed Jul. 29, 2004 by Zhou et al. and also entitled “Quantitative Lateral Flow System and Assay.” The disclosures of these provisional applications are hereby incorporated herein in their entirety by this reference.

### TECHNICAL FIELD

This application generally relates to a qualitative and quantitative assay and system for detecting the presence of at least one analyte in biological samples, particularly samples that contain whole blood, red blood cells, white blood cells, or other cell types, and determining or quantifying the amount of the at least one analyte present.

### BACKGROUND OF THE INVENTION

Many have tried to design a lateral flow assay for determining the presence and quantity of analytes in biological samples, such as blood samples that contain whole blood, red blood cells, or white blood cells, but have failed. The reasons for failure are many but may be attributable primarily to factors such as hemolysis of the red blood cells creating high background noise, low filtering efficiency, for example, resulting in leakage of the red blood cells onto the chromatographic strip, requirement for a relatively large sample volume (such as requiring 100 μl of sample or more), low efficiency in dissolving a conjugate or detectable agent, volume variation because of variation in cell volume when cells are present, long assay time, and inefficiency in dissolving the conjugate for detection of the analyte. It would be desirable to design a lateral flow assay and system that can overcome one or more of these problems in the prior art.

In addition, it would be desirable to simplify the structure of the test strips for lateral flow assays to improve efficiency of the assay and to reduce manufacturing cost.

U.S. Pat. No. 6,136,610 to Polito et al. describes a method and apparatus for performing a lateral flow assay. U.S. Pat. No. 6,528,323 to Thayer et al. describes a bidirectional lateral flow test strip and method for conducting a lateral flow assay. While the methods and system described in these patents are useful for detecting and quantifying most analytes, these patents do not teach how the methods and system can be used to analyze samples containing cells, including red blood cells and/or white blood cells or other cell types. PCT Published Patent Application No. WO 03/008933 describes a test strip for conducting a lateral flow assay for a sample containing whole cells. However, the test strip in WO 03/008933 can be improved to simplify the structure, improve efficiency, reliability, reduce volume dependency and reduce manufacturing cost.

Different strategies have been applied to remove cells, such as red blood cells, from samples, such as blood samples, for detection of analytes, such as infectious disease organisms or antibodies to the infectious disease organisms. However, until now, few strategies have worked well. For example, U.S. Pat. No. 5,766,552 to Doshi et al. discloses the use of a porous material such as an absorbent pad which contains a mixture of both free agglutinating agents and particle-associated agglutinating agents intimately associated with nucleating particles. This filtering system requires about 100 μl of whole blood as shown in FIG. 4 therein.

Human erythrocytes contain on their cell surfaces several transmembrane proteins that may be suitable targets for making antibodies to red blood cells. For example, Band 3 is associated with the electroneutral exchange of chloride and bicarbonate across the cell membrane. Band 3 is a 911 amino acid glycoprotein having a 43 kDa amino-terminal cytosolic domain that binds the cytoskeleton, hemoglobin and glycolytic enzymes, and a 52 kDa carboxyl-terminal membrane domain that mediates anion transport, as described in Wang, D. N. (1994).

Two peptides of Band 3 have been purified, C1 containing Ala893-Val911 and KS4 containing Gly647-Arg656, as described in Fu, G. et al. (2004). The C1 peptide was found to contain protease activity, cleaving glycophorin A (GPA) at Leu118-Ser119 in a dose-dependent manner, but the KS4 peptide did not cleave GPA under the same conditions.

Human erythrocytes further contain on their cell surface another protein, glycophorin. Glycophorin A (GPA) has been reported to enhance the expression of Band 3 anion transport activity at the cell surface of Xenopus oocytes. Young, M. T. and Tanner, M. J. (2003). The authors found that the C-terminal cytoplasmic tail of GPA enhanced trafficking of Band 3 to the cell surface, whereas the extracellular residues 68-70 increased the specific anion transport activity of Band 3.

Up to the present, there is lacking a rapid, effective and efficient quantitative lateral flow assay and system that can be used for determination of analytes in biological samples, such as in a blood sample, in a point-of-care setting, or a lateral flow assay and system that can be used for determination of analytes that are present in a small volume of sample, such as from a finger prick, or a lateral flow assay and system that can be used for determination of analytes that is volume independent, or that would address other problems in the prior art lateral flow assays and systems.

### SUMMARY OF THE INVENTION

It is, therefore, one of the objects of the present invention to provide solutions to the problems faced by the prior art lateral flow assays and systems for determination of analytes in biological samples, including but not limited to samples containing cells, such as red blood cells or white blood cells, or other cell types.

It is another one of the objects of the present invention to provide a lateral flow assay and system, including a test strip and/or a cassette for holding the test strip, that is quantitative.

It is another one of the objects of the present invention to provide a lateral flow assay and system as above that is volume independent.

It is another one of the objects of the present invention to provide a lateral flow assay and system as above that can be performed using small volume of samples, such as in the range of less than about 100 μl. Typically, the sample volume is less than about 90 μl. More typically, the sample volume is less than about 80 μl. Preferably, the sample volume is less than about 70 μl. More preferably, the sample volume is less than about 60 μl. Still more preferably, the sample volume is less than about 50 μl. Most preferably, the sample volume is about 40 μl.

It is a further one of the objects of the present invention to provide a lateral flow assay and system as above that is efficient in dissolving the conjugate or detectable agent.

It is yet another one of the objects of the present invention to provide a lateral flow assay and system as above that provides good filtering for cells, such as red blood cells.

In accordance to one of the objects of the invention, there is provided an invention as follows.

In general, one embodiment of the invention comprises a test strip for a lateral flow assay for detection of at least one analyte in a sample containing a fluid comprising:

(1) a first membrane, wherein the first membrane comprises a sample filter and the sample filter comprises a first pore size and, optionally, a first agglutinating agent;

(2) optionally, a second membrane, wherein the second membrane comprises a first fluid collector and the first fluid collector comprises a second pore size, wherein the second membrane, if present, is in capillary contact with the first membrane;

(3) optionally, a third membrane, wherein the third membrane comprises a conjugate pad and the conjugate pad, if present, is in capillary contact, directly or indirectly, with the chromatographic strip, and wherein the conjugate pad comprises at least one mobilizable detectable agent, and the at least one mobilizable detectable agent is a first mobilizable detectable agent;

(4) a fourth membrane, wherein the fourth membrane comprises a chromatographic strip that comprises a first end and a second end, at least one capture band, at least one control band that optionally comprises a control agent and, optionally, at least one mobilizable detectable agent, wherein the at least one mobilizable detectable agent is a second mobilizable detectable agent, wherein the at least one capture band comprises an immobilized capture agent for capturing the at least one analyte, wherein the chromatographic strip allows lateral flow of fluid from the first end to the second end or from the second end to the first end, and wherein the chromatographic strip is in capillary contact with at least one of the first, second or third membrane, directly or indirectly;

(5) optionally, a fifth membrane comprising a buffer pad for application of sample, buffer, or reagent, wherein the fifth membrane, if present, is in capillary contact with the fourth membrane and optionally comprises a second agglutinating agent;

(6) a sixth membrane, wherein the sixth membrane comprises a first absorbent pad, and the first absorbent pad is in capillary contact with the chromatographic strip directly or indirectly;

(7) optionally, a seventh membrane, wherein the seventh membrane comprises a second absorbent pad, and the second absorbent pad, if present, is in capillary contact with the sixth membrane; and

(8) optionally, an eighth membrane, wherein the eighth membrane comprises a second fluid collector, and the second fluid collector, if present, is in capillary contact with the fourth membrane and the fifth membrane, if present; and

wherein the test strip is configured to allow detection or quantitation of the at least one analyte in the sample, and the sample contains red blood cells.

Typically, the test strip comprises the second membrane and the ratio of the second pore size to the first pore size is less than about 20 and is greater than about 1.

In one alternative, the test strip comprises the second membrane and wherein at least a portion of the first membrane is situated on top of the second membrane.

In another alternative, the test strip comprises the second membrane and the conjugate pad, and the first membrane is in capillary contact with the conjugate pad through the second membrane but does not physically touch the conjugate pad.

In still another alternative, the test strip comprises the fifth membrane, and the fifth membrane comprises a second agglutinating agent.

In yet another alternative, the first absorbent pad is in capillary contact with the fifth membrane, directly or indirectly.

Another embodiment of the invention comprises a test strip for a lateral flow assay for detection of at least one analyte in a sample containing a fluid comprising:

(1) a first membrane comprising a sample filter, wherein the sample filter optionally comprises an agglutinating agent;

(2) a second membrane comprising a chromatographic strip, wherein the chromatographic strip includes a first end and a second end, at least one capture band that comprises an immobilized capture agent for capturing the at least one analyte, at least one control band and, optionally, a first mobilizable detectable agent, wherein the chromatographic strip supports lateral flow of fluid from the first end to the second end or from the second end to the first end, and wherein the chromatographic strip is in capillary contact with the sample filter;

(3) optionally, a third membrane comprising a conjugate pad, wherein the conjugate pad comprises a second mobilizable detectable agent, and the conjugate pad, if present, is in capillary contact with the chromatographic strip;

(4) a fourth membrane comprising a buffer pad, wherein the buffer pad is in capillary contact with the conjugate pad or the chromatographic strip;

(5) a fifth membrane comprising a first absorbent pad;

(6) optionally, a sixth membrane comprising a second absorbent pad; and

(7) optionally, a seventh membrane comprising a first fluid collector;

wherein the test strip is configured to allow detection or quantitation of the at least one analyte in the sample, and the sample contains red blood cells.

Yet another embodiment of the invention comprises a test strip for a lateral flow assay for detection of at least one analyte in a sample comprising:

(1) a chromatographic strip having a first end and a second end, the test strip including a capture band for capturing the analyte;

(2) a fluid-transmitting element in operable contact with the first end of the chromatographic strip, the fluid-transmitting element being selected from the group consisting of a sample pad and a first sample filter, the fluid-transmitted element being located so that fluid applied to the fluid-transmitting element passes through the fluid-transmitting element and is applied to the chromatographic strip;

(3) at least one absorbent pad in operable contact with the fluid-transmitting element;

(4) optionally, a conjugate pad in operable contact with the second end of the chromatographic strip, the conjugate pad including a labeled specific binding partner for the analyte;

(5) a fluid collector in operable contact with either the conjugate pad, if present, or with the second end of the chromatographic strip, if the conjugate pad is not present, so that fluid applied to the fluid collector passes through the fluid collector to the conjugate pad, if present, or to the second end of the chromatographic strip if the conjugate pad is not present;

(6) a second sample filter in operable contact with the fluid collector so that liquid passing through the second sample filter is applied to the fluid collector; and

(7) optionally, a backing in contact with one side of the chromatographic strip, the backing being situated so that fluid can pass unimpeded from the fluid-transmitting element in operable contact with the first end of the chromatographic strip and from the fluid collector or conjugate pad in operable contact with the second end of the chromatographic strip into the chromatographic strip.

Yet another embodiment of the invention comprises a test strip for a lateral flow assay for detection of at least one analyte in a sample comprising:

(1) a chromatographic strip having a first end and a second end, the test strip including a capture band for capturing the analyte;

(2) a first sample filter in operable contact with the first end of the chromatographic strip, the first sample filter being located so that fluid applied to the first sample filter passes through the first sample filter and is applied to the chromatographic strip;

(3) at least one absorbent pad in operable contact with at least part of the first sample filter so that the at least one absorbent pad can withdraw fluid from the chromatographic strip at the first end of the chromatographic strip, the fluid being drawn back through the sample filter;

(4) optionally, a conjugate pad in operable contact with the second end of the chromatographic strip, the conjugate pad comprising a mobilizable labeled specific binding partner for the analyte;

(5) a fluid collector in operable contact with either the conjugate pad, if present, or with the second end of the chromatographic strip, if the conjugate pad is not present, so that fluid applied to the fluid collector passes through the fluid collector to the conjugate pad, if present, or to the second end of the chromatographic strip if the conjugate pad is not present;

(6) a second sample filter in operable contact with the fluid collector so that liquid passing through the second sample filter is applied to the fluid collector; and

(7) optionally, a backing in contact with one side of the chromatographic strip, the backing being situated so that fluid can pass unimpeded from the first sample filter in operable contact with the first end of the chromatographic strip and from the fluid collector or conjugate pad in operable contact with the second end of the chromatographic strip into the chromatographic strip.

Yet another embodiment of the invention comprises a test strip for a lateral flow assay for detection of at least one analyte in a sample comprising:

(1) a chromatographic strip comprising a first end and a second end, at least one capture band comprising an immobilized capture agent for capturing the at least one analyte, and at least one control band comprising an immobilized control agent for determination of non-specific binding;

(2) a conjugate pad, wherein the conjugate pad is in capillary contact with the second end of the chromatograph strip, and wherein the conjugate pad comprises a mobilizable detectable agent that is capable of binding to the at least one analyte or to the capture agent after capturing the analyte;

(3) a sample filter that is adjacent to the conjugate pad on the side closer to the second end, wherein the sample filter optionally comprises an agglutinating agent, and the sample filter is in capillary contact with the chromatographic strip;

(4) optionally a fluid collector that, if present, is situated between the sample filter and the chromatographic strip;

(5) optionally, a buffer pad situated at the first end of the chromatographic strip and is in capillary contact with the chromatographic strip;

(6) a first absorbent pad situated at the first end of the chromatographic strip that is in capillary contact with the chromatographic strip, either directly or indirectly; and

(7) optionally, a second absorbent pad that, if present, is in capillary contact with the first absorbent pad; wherein the test strip allows detection or quantitation of an analyte in a sample containing whole cells.

Additional objects, features, or advantages of the present invention will be set forth in part in the description that follows, and in part will be apparent to a person of ordinary skill in the art upon reading the description herein or may be learned by practicing the invention. The objects and advantages of the invention will be realized and attained by means of the elements and combinations particularly pointed out in the Summary of the Invention and the appended claims. Moreover, advantages described in the specification, if not included in the claims, are not per se limitations to the claimed invention.

The inventions illustratively described herein pan suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

### BRIEF DESCRIPTION OF THE DRAWINGS

FIG. 1 is a top plan view of an example of a cassette for holding the test strip of the present invention.

FIG. 2 is a side view of one embodiment of a test strip of the present invention where a sample is applied at Port-1.

FIG. 3 is a side view of another embodiment of a test strip of the present invention where a sample is applied at Port-2.

FIG. 4 is a side view of a further embodiment of the test strip of the present invention where a sample can be applied at both Port-1 and Port-2.

FIG. 5 is a side view of one embodiment of the test strip of the present invention and a top plan view of a cassette that may be used with the test strip, showing correspondence between the test strip and portions of the test strip that are visible in the cassette.

FIG. 6 is a top view of one embodiment of the present test strip showing bidirectional flow of fluid upon application of sample at Port-1 and buffer at Port-2.

FIG. 7 is a top view of another embodiment of the present test strip showing bidirectional flow of fluid upon application of sample at both Port-1 and Port-2.

FIG. 8 is a side view of an alternative embodiment of the test strip generally similar to that of FIG. 3 but one in which the sample reacts with conjugate before reaching the sample filter, at least for sample applied to the conjugate pad, typically through Port-2.

FIG. 9 is a side view of an alternative embodiment of the test strip generally similar to that of FIG. 4 but one in which the sample reacts with conjugate before reaching the sample filter, at least for sample applied to the conjugate pad, typically through Port-2.

FIG. 10 is a side view of another alternative embodiment of a test strip generally similar to that of FIG. 3, but one in which, stacked atop the chromatographic medium at the second end of the chromatographic medium, are a conjugate pad, a sample filter, and a fluid collector, with fluid being applied to the conjugate pad and the fluid collector being in contact with the chromatographic medium.

FIG. 11 is a side view of another alternative embodiment of a test strip generally similar to that of FIG. 3, but one in which, stacked atop the chromatographic medium at the second end of the chromatographic medium, are a conjugate pad, a sample filter, and a fluid collector, with fluid being applied to the conjugate pad and the fluid collector being in contact with the chromatographic medium.

FIG. 12 is a detailed side view of an embodiment of a test strip capable of performing an indirect assay for human hepatitis C virus (HCV) using gold anti-DNP antibody and DNP-BSA as a control.

FIG. 13 is a detailed side view of an embodiment of a test strip capable of performing a sandwich assay for prostate specific antigen (PSA) using gold anti-DNP antibody and DNP-BSA as a control.

FIG. 14 is a detailed side view of an embodiment of a test strip capable of performing a sandwich assay for antibody specific for human HIV using gold anti-DNP antibody and DNP-BSA as a control.

FIG. 15 is a detailed side view of an embodiment of a test strip capable of performing an indirect assay for antibody specific for human HIV using gold anti-DNP antibody and DNP-BSA as a control.

FIG. 16 is a detailed side view of an embodiment of a test strip capable of performing an indirect assay for antibody specific for human HIV and for antibody specific for HCV using gold anti-DNP antibody and DNP-BSA as a control.

FIG. 17 is a detailed side view of an embodiment of a test strip capable of performing a sandwich assay for hepatitis B surface antigen (HBsAg) and for Treponema pallidum antigen using gold anti-DNP antibody and DNP-BSA as a control.

### DETAILED DESCRIPTION OF THE INVENTION

The inventors herein have discovered a lateral flow assay method and system including a test strip and/or a cassette for holding the test strip, for determination of the presence and/or quantity of analytes in samples, including but not limited to biological or other samples containing materials including antigens, antibodies, hormones and other secreted proteins, cell surface proteins, transmembrane proteins, glycoproteins, enzymes, proteins associated with cells and other proteins, proteins associated with pathogens such as bacteria, viruses, and fungi, carbohydrates, drugs, peptides, toxins, nucleic acids, small molecules, and aptamers. This novel assay or system can detect and/or quantitate analytes in small volumes of samples. Generally, the sample volume is less than about 100 μl. Typically, the sample volume is less than about 90 μl. More typically, the sample volume is less than about 80 μl. Preferably, the sample volume is less than about 70 μl. More preferably, the sample volume is less than about 60 μl. Still more preferably, the sample volume is less than about 50 μl. Most preferably, the sample volume is about 40 μl. This assay or system can also separate cells from fluid in a sample, such as red blood cells or white blood cells or other cell types. This assay or system is substantially volume independent such that, for example, the results are consistent regardless of variation in cell volume, such as red blood cell volume, of red blood cells present in the sample. The assay or system also provides low background noise and is highly efficient.

For use herein, an “analyte” refers to the material to be detected by use of the lateral flow test strip and method of the present invention. “Analyte” includes but is not limited to: antigens, antibodies, hormones (such as TSH, hCG, LH), drugs, cardiac markers (such as Troponin I, creatine kinase—MB isoforms (CKMB), myoglobin, C-reactive protein (CRP), fatty acid binding protein (FABP), glycogen phosphorylase isoenzyme BB (GPBB), B-type natriuretic peptide (BNP), and pro-BNP), autoimmune disease markers, tumor markers (such as PSA, CEA, α-fetoprotein), proteins associated with a cell (“cell proteins”), secreted proteins, enzymes, cell surface or transmembrane proteins, glycoproteins and other proteins, proteins or carbohydrates associated with pathogens, such as bacteria, viruses, or fungi, peptides, toxins, nucleic acids, aptamers and carbohydrates. Analytes, as used herein, further includes molecules detectable by specific non-antibody binding proteins such as receptors and nucleic acids detectable by specific Watson-Crick base pairing (hybridization). Other analytes are described further throughout the specification.

An “analyte binding agent” herein is a molecule that specifically binds an analyte in a sample to be analyzed. The “analyte binding agent” may be an antibody or an antigen but is not limited to such. “Analyte binding agent” includes engineered proteins, peptides, haptens and lysates containing heterogeneous mixture of antigens having analyte binding sites. In one typical, but not exclusive embodiment, the analyte binding agent is either an antibody for binding to an antigen in a sample to be analyzed or is an antigen for binding to an antibody in the sample to be analyzed. If the analyte is a nucleic acid molecule, the analyte binding agent can be a nucleic acid molecule that binds specifically to it such as by Watson-Crick base pairing, or can be a protein that binds a nucleic acid sequence on the basis of sequence-specific interactions.

The term “antigen” as used herein includes infectious agents and other microorganisms or portions thereof, such as bacteria, viruses, capsids, nucleocapsids, or other portions of viruses, fungi, prions, or parasites. The analyte of interest preferably contains an immunogenic portion such that antibodies can be raised against that portion for detection purposes. Bacteria include Gram positive and Gram negative bacteria such as, for example, Bacillus anthracis, Escherichia coli, Salmonella species, Shigella species, Pasteurella pestis, Helicobacter pylori, Vibrio cholerae, Staphylococcus species, etc. Viruses include HIV, hepatitis virus A, B, C and D, Herpes simplex virus, cytomegalovirus (CMV), Ebola virus, papilloma virus such as HPV, Rhinoviruses including influenza viruses, SARS virus, and Vaccinia viruses. “Antigen” also includes an immunogenic portion of any compound or infectious agent to which an antibody can be raised. Additionally, the term “antigen” can also include antibodies that are to be detected or macromolecules that can raise antibodies. For example, in testing for human immunodeficiency virus (HIV) or hepatitis C virus (HCV), human anti-HIV antibodies or anti-HCV antibodies are the antigens to be detected, such as by anti-human IgG. In the case of human autoimmune diseases, such as rheumatoid arthritis, Hashimoto's thyroiditis, systemic lupus erythematosus, and other conditions characterized by an abnormal antibody response to autoantigens, the human antibodies against such autoantigens become the antigen.

The term “antibody” as used herein includes polyclonal or monoclonal antibodies or fragments that are sufficient to bind to an antigen or an analyte of interest. The antibody fragments can be, for example, monomeric Fab fragments, monomeric Fab′ fragments, or dimeric F(ab)′₂ fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. However, in most cases, such modifications are not required to generate an antibody that is suitable for use with the present invention.

The term “capture band” as used herein refers to a region or zone on the chromatographic strip that contains at least one analyte binding agent. The analyte binding agent is usually immobilized in a band or zone such that after reaction with a detectable agent, the band or zone produces an observable or measurable result reflecting the presence or amount of analyte present in the sample. The “capture band” may be comprised of more than one capture zone for capturing more than one analyte in the sample, in which event, more than one analyte binding agent may be used. For example, two assay combinations that are considered to be within the scope of the invention are assay combinations that simultaneously detect hepatitis C virus (HCV) and human immunodeficiency virus (HIV), and assay combinations that simultaneously detect Hepatitis B surface antigen (HBsAg) and Treponema pallidum antigen (TP). Still other combinations are possible and are within the scope of the invention.

The term “conjugate” and “detectable agent” are used interchangeably herein to refer to an antibody or an antigen that is conjugated to a detectable material such as a colored agent, a fluorescent agent or a chemiluminescent agent. In the practice of the present invention, the “conjugate” or “detectable agent” specifically binds the analyte to be determined or the captured analyte immobilized on the capture band. Optionally, the “conjugate” or “detectable agent” produces a measurable quantitative reading at the capture band that reflects the amount of an analyte present at the capture band. As described further below, the direct measurable quantitative density in the capture band does not necessarily reflect the amount of an analyte present at the capture band through binding, but the RI (relative density) does reflect the amount of an analyte present at the capture band.

The “detectable material” as used herein refers to any material that can be conjugated to an antigen or an antibody and that can be detected, such as at the capture band. The material can be a particle, a colored material, a fluorescent material, a chemiluminescent material and may include more than one material. If more than one material is used, any combination of the possible materials can be used. For example, if the assay is intended to detect more than one analyte, detectable materials to be used may be fluorescent materials that fluoresce at different wavelengths. The particles can be colloidal gold particles, colloidal sulfur particles, colloidal selenium particles, colloidal barium sulfate particles, colloidal iron sulfate particles, metal iodate particles, silver halide particles, silica particles, colloidal metal (hydrous) oxide particles and the like as described in U.S. Pat. No. 6,136,610, with or without an organic or inorganic coating, protein or peptide molecules, liposomes, or organic polymer latex particles such as polystyrene latex beads. The size of the particles may be related to porosity of the chromatographic strip.

The term “control band” as used herein contains control agents immobilized in control binding zones. The control agents bind specifically to control binding agents to form a control binding pair, as described in U.S. Pat. No. 6,136,610, incorporated herein by this reference. The present invention typically includes two control bands, although the use of two control bands is not required. The two control bands may be the same or different. A particular advantage to having control binding pairs is that they act as internal controls, that is, the control against which the analyte measurement results may be compared on the individual test strip. The controls may be used to correct for strip to strip variability. One of the controls can be designated a high control (“HC”) and the other of the controls can be designated a low control (“LC”). The ratio of HC to LC is typically predetermined as one of the internal quality controls when two controls are used. Additionally, the reflection density of HC, or, alternatively, of LC, can be used to determine the RI (relative density) of the testing band (analyte). The standard curve is made for any quantitative assays by the RI of standard reagents with serial concentrations. In qualitative assays, the quantitation is measured by the ratio of the RI of Signal/Cutoff (S/C) while the cutoff is determined by a large number of negative samples. Although, in general, any conventional controls can be used herein, it is generally preferred to use as controls compounds that do not exist in the sample or do not immunologically cross-react with compounds that exist in the sample; for example, 2,4-dinitrophenylated bovine serum albumin (BSA-DNP), which can be purchased from Molecular Probes (Eugene, Oreg., cat# A-23018) can be used as the control reagent. The compound 2,4-dinitrophenol (DNP) is a small molecule which does not exist within the human body but acts as a hapten; that is, it is immunogenic when conjugated to a larger molecule such as a protein carrier and injected into an antibody-producing mammal such as a mouse, a rat, a cow, a rabbit, a horse, a sheep, or a goat.

The term “operable contact” is used herein as follows: Two solid components are in operable contact when they are in contact, either directly or indirectly, in such a manner that a liquid can flow from one of the two components to the other substantially uninterruptedly, by capillarity or otherwise. “Direct contact” means the two elements are in physical contact, such as edge-to-edge or front-to-back. “Indirect contact” means the two elements are not in physical contact, but are bridged by one or more conducting means. This bridging by one or more conducting means could be either edge-to-edge or front-to-back. The term “capillary contact,” used herein, is equivalent to operable contact.

A “direct assay” for detection of an analyte, such as for PSA (prostate specific antigen) or TSH (thyroid stimulating hormone), for example means a sandwich assay. For example, for an assay of PSA, labeled anti-PSA antibody reacts with PSA present in the sample to form a complex. This complex is detected at the capture band where either PSA antigen or anti-PSA could be coated. If the antigen is aggregated or contains multiple copies of the same epitope (antigenic determinant), and it is desired to bind an antibody to the test strip at the capture band, the first and second antibodies specific for the analyte can be identical. Otherwise, the first and second antibodies are two antibodies that bind to different epitopes on the analyte. In a sandwich assay, the sample could be added in both Port-1 and Port-2 of the device as shown below. Optionally, the sample could be added only in Port-1, in an assay utilizing a unidirectional lateral flow to provide a signal.

In an “indirect assay” for anti-HIV antibody, for example, the sample is added to Port 1 and, as the sample flows down the chromatographic strip over the test band, anti-HIV antibodies in the sample bind to HIV antigens in the test band. When buffer is added to Port 2, it solubilizes an anti-human IgG-gold conjugate which flows up the chromatographic strip and binds to human antibodies which specifically bound to the HIV antigens in the test band. In this format of an indirect assay, the sample is always added in Port 1 of the device as shown below.

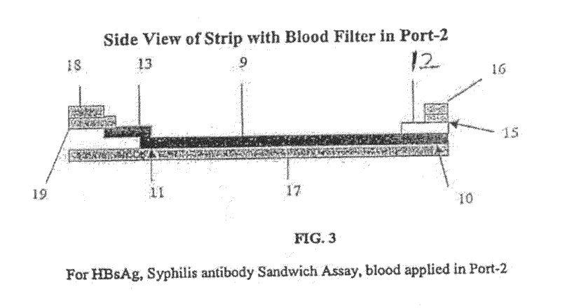

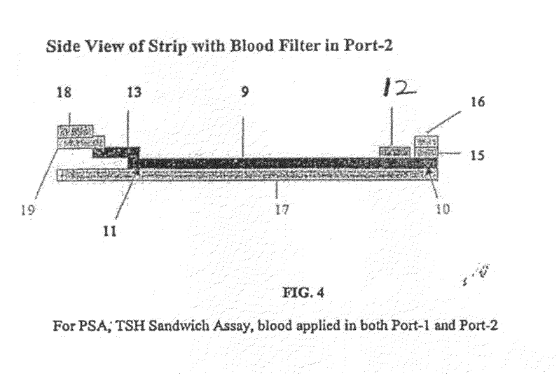

A “stop flow” assay as used herein is an assay in which flow in the first direction stops. In stop flow, the liquid (sample or buffer) that is added in Port-1 is added in a relatively small volume so that there is not enough liquid to flow through the nitrocellulose membrane, and flow stops before the flow reaches the labeled reagent (conjugate). The purpose of performing a stop flow assay is to prewet the nitrocellulose membrane, to block some non-specific protein binding sites, and to ensure that chemicals on the surface of the nitrocellulose membrane are evenly distributed before labeled reagents flowed into these areas. This is to be contrasted with a “reversed flow” assay. In a reversed flow assay, a larger volume of liquid is added to Port-1 so that this liquid could flow back. For a reversed flow assay, the cassette is constructed so that when the correct volume of sample is added to Port 1, the liquid moves down the strip toward Port 2 past the control and test zones and then stops and then reverses flow back towards the absorbent pad. Sample is then added to Port 2 and flows up the strip toward the absorbent pad. Additionally, a bidirectional flow assay format can be performed using the devices of FIGS. 3 and 4 according to the present invention. In a bidirectional flow assay format, liquid is added sequentially to both ends of the nitrocellulose membrane or other chromatographic medium, typically through Port-1 and Port-2, and flow within the nitrocellulose membrane occurs in both directions.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further the terminology used to describe particular embodiments is not intended to be limiting, since the scope of the present invention will be limited only by its claims.

Further, while the present invention may be used independently or in conjunction with any analytical device adapted to read the results manually or automatically, the invention herein is exemplified using the apparatus and cassette of U.S. Pat. No. 6,136,610 and, in one embodiment of the invention, utilizing the bidirectional flow mechanism of U.S. Pat. No. 6,528,323. It is to be understood that the present invention is not limited to use in such apparatus or cassette.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claim, the singular forms include the plural forms. For example the terms “a,” “an,” and “the” include plural references unless the content clearly dictates otherwise. Hence, unless otherwise indicated, reference to “a sample filter,” includes one or more sample filters. Optionally, a buffer pad can be situated above any sample filter or assemblage of sample filters. This applies to all embodiments described below.

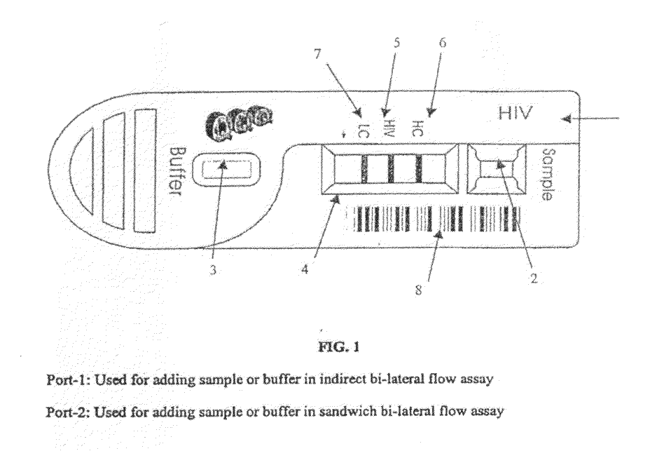

Referring to FIG. 1, FIG. 1 is a top plan view of a prior art cassette (1) that can be used with the test strip of the present invention: the cassette (1) has two ports. Port-1 (2) can be used for application of a sample in a sandwich assay, such as detection of HBsAg; or an indirect bilateral flow assay for detection of an analyte, such as anti-HIV antibody; and Port-2 (3) can be used for application of a sample in a sandwich assay, or a reagent, such as a buffer in an indirect bilateral flow assay. The cassette (1) also contains a testing window (4) for viewing results of the assay. Through the test window (4), the capture band (5) for the analyte to be detected, labeled human anti-HIV in this example, can be observed, together with a first control band (6), labeled HC in this example, and a second control band (7), labeled LC in this example. The cassette (1) can optionally include a bar code (8) for management of assay types, the product expiration date, and adjustment of inter-lots variables.

In the present invention, if the cassette of FIG. 1 is used, Port-1 (2) or Port-2 (3) can each be used for application of sample or reagent, as described in greater detail below.

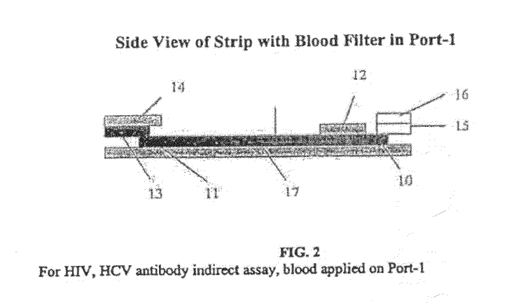

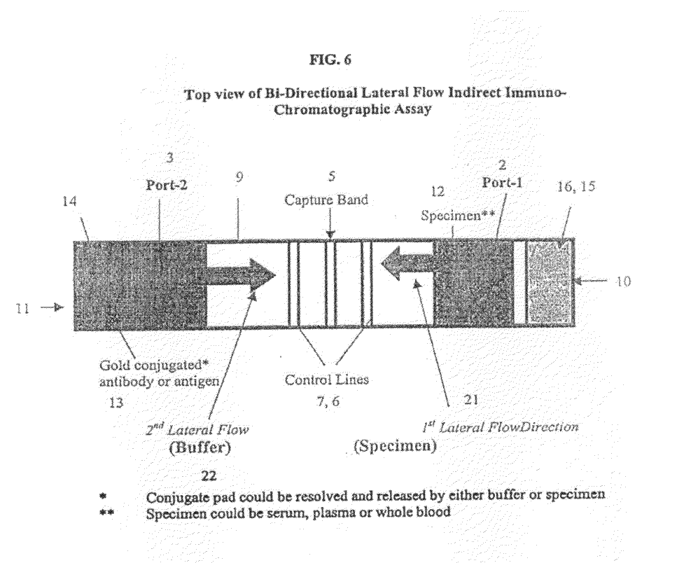

Referring to FIG. 2, FIG. 2 is a side view of a test strip for use in one embodiment of the present invention. In this embodiment, shown in FIG. 2, there is a sample filter (12) situated at Port 1 (2 of FIG. 1) at a first end (10) of a chromatographic strip (9), and a buffer pad (14) situated at Port 2 (3 of FIG. 1). The buffer pad (14) sits on top of a conjugate pad (13) which contains at least one mobilizable detectable agent, commonly referred to as a “conjugate,” that specifically binds the analyte or an agent that binds the analyte. Such a conjugate may be, for example, an analyte-specific antibody or analyte-specific antigen conjugated to colloidal gold, for example. Optionally, the conjugate pad (13) also contains a second mobilizable detectable agent that specifically binds an immobilized control agent at one or more of the control bands (6, 7), as shown in FIG. 6, for example. For a whole blood assay, the sample filter (12) contains an agglutinating agent to agglutinate the red blood cells in the whole blood sample. A first absorbent pad (15) is situated at the first end (10), adjacent to the sample filter (12) on the side of the sample filter (12) away from the conjugate pad (13) or buffer pad (14). An optional second absorbent pad (16) in capillary contact with the first absorbent pad (15) may be used. The second absorbent pad (16), if present, is situated directly on top of the first absorbent pad or overlaps the first absorbent pad (15). The first absorbent pad (15) is in capillary contact with or overlaps the first end (10) of the chromatographic strip (9). A plastic backing pad (17) can optionally be used to support the chromatographic strip. The chromatographic strip (9) also contains at least one capture band (5) for each analyte to be detected and one or more control bands (6, 7) as shown in FIG. 6, for example. Each capture band (5) contains an immobilized antibody or an antigen that specifically reacts with the analyte to be detected in the sample. Each control band (6, 7) optionally contains an immobilized antibody or antigen that reacts non-specifically with the sample, or reacts specifically with a control reagent in the conjugate pad (13).

In performing an indirect assay using the format of FIG. 2, a sample containing whole blood is applied to the sample filter (12) at Port 1 (2), as shown in FIG. 6. RBCS are retained in the sample filter (12) while fluid from the sample flows from the sample filter (12) to the chromatographic strip (9) at the first end (10), and from the first end (10) in a first lateral flow direction (21 of FIG. 6) towards the second end (11). In the course of the first fluid flow, the sample fluid moves past the capture band (5) which contains, for example, HIV antigen or HCV antigen in an HIV or HCV test, respectively, that interacts with the analyte in the sample, such as human anti-HIV antibody or human anti-HCV antibody, respectively. The fluid also moves past the control bands (6, 7) during the course of the first lateral fluid flow (21). Further, the fluid flow in the first lateral flow direction (21) desirably and apparently ceases flow between the control band closest to the conjugate pad (7) and the conjugate pad (13). The analyte in the sample, if present, is primarily captured at the capture band (5) during the course of fluid flow in the first lateral flow direction (21), forming a first immunocomplex, such as a HIV-human anti-HIV antibody complex, at the capture band (5). Some analyte and other antibodies present in the whole blood sample may bind non-specifically at the one or more control bands (6, 7). A buffer is then applied to the buffer pad (14) to release the conjugate in the conjugate pad (13). In one embodiment, the released conjugate contains at least one and optionally two labeled reagents, one that specifically reacts with the first immunocomplex at capture band (5), for example, a labeled anti-human IgG and, optionally, one that reacts with the control reagent at the control bands (6, 7). The released conjugate migrates from the second end (11) of the chromatographic strip (9) in the second lateral flow direction (22) towards the first end (10). During fluid flow in the second lateral flow direction (22), a detectable complex of labeled control binding reagent and control reagent is formed at the control bands (6 and 7) and a detectable second immunocomplex of labeled anti-human IgG antibody and the first immunocomplex is formed at the capture band (5). The indirect assay therefore allows the first immunocomplex, formed between analyte in the sample and the capture reagent at the capture band (5) during the first flow, to be detected during the second flow.

Notably, when strip configuration of FIG. 2 is used for determination of analytes in non-whole blood samples, such as serum or plasma samples, the sample filter (12) need not contain an agglutinating agent. This configuration can be used for both sandwich assays and/or indirect assays. In a sandwich assay, for example, such as a HBsAg test, a sample or a buffer, if the best performance requires such. (In the case of a sandwich assay of HBsAg, the serum sample could be added in both Port 1 and Port 2; it also could be added in only Port-2.) The distinction between indirect and sandwich assays, as performed in test strips according to the present invention, is as follows. The definition for an indirect assay is that the labeled reagent does not react with the analyte directly but reacts with the immunocomplex. For example, if the sample was added in Port-2, in HBsAg testing, one anti-HBsAg antibody was coated in capture band, another anti-HBsAg was labeled and coated in conjugate pad, the first immunocomplex was formed when the sample added to Port-2, then captured in testing band to form the sandwich, both immobilized and labeled reagents are the same antibody or antigen (even with different epitopes). On the other hand, in an indirect Assay such as HIV or HCV antibody testing, the immobilized reagent (HIV or HCV antigen) in capture band and the labeled reagent (anti-human IgG) in conjugate pad are not the same reactant. The immobilized capture reagent reacts with analyte directly, and the labeled reagent reacts with the complex of analyte and captured reagent. Sample is applied to the sample filter (12) at Port 1(2), as shown in FIG. 6. Fluid will flow from the sample filter (12) to the chromatographic strip (9) at the first end (10) and from the first end (10) in a first lateral flow direction (21 of FIG. 6) toward the second end (11), past the capture band (5) and the control bands (6, 7); the fluid ceases flow (the “Stop Flow” format) before reaching the conjugate pad (13) or optionally, the fluid can flow into the conjugate pad (13) and dissolve the conjugate, if it is desired for improved performance of the assay. Typically, in assays performed with test strips according to the present invention, particularly indirect assays, it is preferred to have a stop flow format, i.e. not allowing the liquid to flow through and reach the conjugate, especially for indirect assays (antibody testing) because the antibodies in the sample will interact with the labeled anti-human IgG (or IgM) in conjugate, forming an immunocomplex before the conjugate reached capture band. This causes a false negative result. Therefore, in an indirect assay, the liquid from Port 1 could not reach the conjugate. However, in a sandwich assay, the use of the stop flow format is not required.

Thus, in one embodiment, between the control band (7) and the conjugate pad (13), fluid from the sample ceases flow in the first lateral flow direction (21). If the analyte being detected is present in the sample, HBsAg, for example, it is captured at the capture band (5) which contains, for example, an immobilized human anti-HBsAg antibody (“immobilized capture antibody”). In the course of fluid flow in the first lateral flow direction, a first immunocomplex, such as HBsAg and anti-HBsAg capture antibody complex is formed at the capture band (5). Again, during the course of fluid flow in the second lateral flow direction (22), if there is any unbound analyte remaining in the sample after the first flow, additional first immunocomplex is formed during the second flow.

A second aliquot of the sample is applied to the buffer pad (14) which, in this format, contains an RBC agglutinating agent. Cells in the sample are retained by the buffer pad and fluid from the sample flows through the conjugate pad (13) and releases the conjugate. The conjugate contains a first labeled mobilizable reagent that reacts with the analyte in the sample, such as labeled human anti-HBsAg antibody conjugated to a detectable agent, for example, colloidal gold. Optionally, the conjugate contains a second labeled mobilizable reagent that reacts with the control reagent at the control bands (6, 7). During fluid flow in the second lateral flow direction (22), a second immunocomplex, such as labeled human anti-HBsAg antibody and HBsAg, forms and migrates from the second end (11) of the chromatographic strip (9) in the second lateral flow direction (22) towards the first end (10) if the analyte is present in the sample. As fluid flow in the second lateral flow direction continues, a detectable complex of labeled control-binding reagent and control reagent is formed at the control bands (6 and 7) and a detectable third immunocomplex containing complexed first and second immunocomplexes is formed at the capture band (5). The bidirectional lateral flow aids in washing contaminants away from the capture and control bands, reducing background noise.

The format of FIG. 2 may be used in an indirect assay for detection of analyte in a serum or plasma sample. In this format, the agglutinating agent may optionally be excluded from the sample filter (12). The analysis is conducted by first adding buffer or a liquid reagent to sample filter (12) at Port 1 (2) to prewet the chromatogrphic strip (9). The serum or plasma sample is then added to Port 2 (3). Fluid from the sample releases the conjugate, for example, from the conjugate pad (13). The analyte in the sample, if present, reacts with the conjugate to form a first antigen-antibody binding pair, i.e., a first immunocomplex. The first immunocomplex migrates with the sample fluid in a second fluid flow direction (22) towards the capture band (5) and control bands (6, 7). The first immunocomplex is captured at the capture band (5) in a second antigen-antibody reaction to form a second binding pair, i.e., a second immunocomplex, which can be detected and quantified.

In a variation of the above-mentioned embodiment, not shown, a conjugate pad (13) is not used and mobilizable detectable agents are incorporated into the chromatographic strip (9) at the second end (11). In this embodiment, a buffer pad (14) is situated at Port 2 (3 of FIG. 1), on top of or overlapping with the chromatographic strip (9). The buffer pad (14) may sit on top of the mobilizable detectable agent.

In another variation of the above-mentioned embodiment, not shown, a buffer pad (14) is not used, the conjugate pad (13) is situated on top of and/or overlaps the second end of the chromatographic strip. Buffer can be added directly onto the conjugate pad (13).

In yet another embodiment, shown in FIG. 3, the test strip is suitable for performance of a sandwich assay, such as for detection of HBV surface Ag (HBsAg) and for detection of antibody to Treponema pallidum, causative agent of syphilis. In this format, there is a second sample filter (18), a fluid collector (19), optionally a conjugate pad (13), all situated at the second end (11) of the chromatographic strip (9). There is also a first sample filter (12) and at least one absorbent pad (15) situated at the first end (10) of the chromatographic strip (9). A first absorbent pad (15) is situated on top of the first sample filter (12). FIG. 4 shows a variation in which the first and optionally the second absorbent pad are situated adjacent to the sample filter (12) at the first end (10) of the chromatographic strip (9). The format of FIG. 4 is suitable for performing sandwich assays such as those for prostate specific antigen (PSA) and thyroid stimulating hormone (TSH).

In a variation of the embodiment shown in FIG. 3 (the variation is not shown), a conjugate pad is not used, and the mobilizable detectable agents are incorporated into the chromatographic strip (9) at the second end (11). In this configuration, the fluid collector (19) is directly in capillary contact with the chromatographic strip (9), the fluid collector may be situated entirely on top of the second end (11) of the chromatographic strip (9) or may overlap the chromatographic strip (9).

In another variation of the embodiment of FIG. 3 (the variation is not shown), the first sample filter is replaced with a sample pad for application of a reagent, such as a buffer. The sample pad is composed of an absorbent material which is capable of holding sufficient buffer for running the assay.

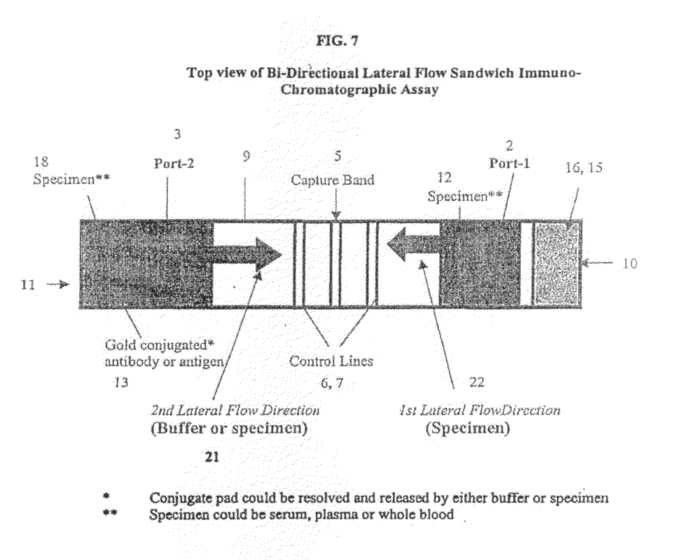

In the operation of an assay using the formats of FIG. 3 and FIG. 4, sample containing whole blood is added to both Port 1 (2) and Port 2 (3) of FIG. 1. In this format, both first sample filter (12) and second sample filter (18) are preferably blood filters to filter out red blood cells. FIG. 7 illustrates the operation of a sandwich assay using the embodiment shown in FIG. 4. An aliquot of a sample containing RBC is applied to the first sample filter (12) at the first end (10) of the chromatographic strip (9). Fluid from the sample flows in the first lateral flow direction (21) from the first end (10) to the second end (11), flowing past the capture band (5) and the control bands (6 and 7). The analyte, if present, is captured at the capture band (5), the analyte-antibody forming a first immunocomplex at capture band (5). A second aliquot of the same sample is then applied to the second sample filter (18) at Port 2 (3). Fluid from the second sample filter (18) passes through a fluid collector (19 of FIG. 4) and a conjugate pad (13) to the second end (11), and then from the second end (11) in a second lateral flow direction (22) toward the first end (10), past the capture band (5) and the control bands (6 and 7). The analyte, if present is captured at the capture band (5) by the detection reagent, such as an antibody, the analyte-antibody complex forming a sandwich. In this format, the analyte, for example, HBsAg, applied to Port 2 (3) will first combine with the conjugate from the conjugate pad, for example, labeled anti-HBsAg antibody, such as anti-HBsAg antibody conjugated to colloidal gold, to form an analyte-conjugate complex, which then migrates to the capture zone and reacts with an anti-HBsAg antibody immobilized at the capture zone, which by that time would have also captured HBsAg from analyte applied to Port 1 (2).

In a variation, addition of sample to sample filter (12) at the first end (10) may be omitted and sample can be added directly to the second sample filter (18) at Port 2 (3). In this format, buffer is first added to Port 1 to prewet the test strip, the buffer flowing in a first lateral flow direction (21) from the first end (10) to the second end (11). Then sample is applied to Port 2 (3). Fluid from sample passes through the fluid collector (19) and a conjugate pad (13) to the second end (11) and from the second end (11) in a second lateral flow direction past the capture band (5) and the control bands (6 and 7). The analyte in the sample combines with the conjugate to form a complex, the complex then flows to the capture band and the control bands. The analyte-conjugate complex is captured at the capture band (5), forming a sandwich.

Notably, when strip configuration of FIG. 3 or FIG. 4 is used for determination of analytes in non-whole blood samples, such as serum or plasma samples, the sample filter (18) in FIG. 3, or the sample filters (12 and 18) in FIG. 4 does not contain an agglutinating agent. This configuration can be used for both sandwich assays and indirect assays.

The sample filter (12) is situated on the chromatographic strip (9) at the first end (10), such that fluid present in a sample, when applied onto the sample filter (12), such as from Port-1 (2) of the cassette of FIG. 1 (1), will flow from the sample filter (12) to the first end (10) of the chromatographic strip (9) and from the first end (10) of the chromatographic strip (9) in a first flow direction toward the second end (11) of the chromatographic strip (9). The sample filter (12) optionally contains an agglutinating agent relevant for removing certain materials, such as cells from a sample. For example, if the sample contains red blood cells, the agglutinating agent may be anti-red blood cell antibodies or may be lectins that agglutinate red blood cells. A sample filter containing such an agglutinating agent or agents is referred to generically as a “whole blood filter” herein. These alternatives are discussed further below.

As used herein, the terms “sample pad” and “sample filter” refer to elements that can be used to receive a sample, such as a sample of blood, serum, or plasma. The term “sample pad” refers to a hydrophilic element, such as a hydrophilic membrane, that can be used to receive a sample. When the sample is or can be whole blood, the sample pad can contain an agglutinating agent as described above. The term “sample filter” can refer to a generally hydrophobic element, such as a glass fiber filter, that can be similarly used to receive a sample. The sample filter can also contain an agglutinating agent as described above. However, the sample filter can be a hydrophilic element, such as a sample pad, pretreated with an anti-erythrocyte antibody or other agglutinating agent. The term “whole blood filter,” as used herein, refers to a sample filter that contains an agglutinating agent. However, when the strip configuration of FIG. 2 is used for determination of analytes in samples other than whole blood samples, such as serum or plasma or other biological fluids, the sample filter (12) need not contain an agglutinating agent. This configuration can be used for both sandwich assays and indirect assays. In a sandwich assay, for example an assay of HBsAg, a sample or a buffer, if the best performance requires such, is applied to the sample filter (12) at Port-1 (2), as shown in FIG. 6. In such a sandwich assay of HBsAg, the sample can be added to both Port-1 and Port-2 or only in Port-2. Fluid will flow from the from the sample filter (12) to the chromatographic strip (9) at the first end (10) and from the first end (10) in a first lateral flow direction (21 of FIG. 6) toward the second end (11), past the capture band (5) and the control bands (6, 7); the fluid ceases flow (the “Stop Flow” format) before reaching the conjugate pad (13) or optionally, the fluid can flow into the conjugate pad (13) and dissolve the conjugate, if it is desired for improved performance of the assay. In an indirect assay, the Stop Flow format is particularly suitable because antibodies in the sample are precluded from interacting with the labeled anti-human IgM or anti-human IgG in the conjugate, forming an immunocomplex before the conjugate reached the capture band and thus giving a false negative result.

In one embodiment of the invention, the conjugate pad (13) is situated at the second end of the chromatographic strip (11), and a buffer pad (14) is situated on top of the conjugate pad (13). In this embodiment, the conjugate pad (13) overlaps the second end (11) of the chromatographic strip (9) by a distance sufficient for fluid to pass from the buffer pad (14) through the conjugate pad (13) and onto the chromatographic strip (9). This distance of overlap may be from about 0.5 mm to about 10 mm, typically from about 1 mm to about 8 mm, preferably from about 2 mm to about 5 mm, and more preferably, about 2-3 mm.

The buffer pad (14) may be of any suitable size provided that it can absorb or hold an amount of fluid sufficient to dissolve the detectable agent in the conjugate pad (13) or in the chromatographic strip (9) as described below. In one embodiment, the buffer pad is larger than the conjugate pad. In another embodiment, the buffer pad is the same size as the conjugate pad. In yet another embodiment, the buffer pad is smaller than the conjugate pad.

In another embodiment of the invention, a conjugate pad (13) is not used, and detectable agents that are usually present in a conjugate pad, when the conjugate pad is used, are incorporated into the second end (11) of the chromatographic strip (9). In such an embodiment, a buffer pad (14) is situated on top of the chromatographic strip (9). In one alternative, the buffer pad (14) overlaps the second end (11) of the chromatographic strip (9) by a distance sufficient to allow fluid applied on the buffer pad (14) to flow by capillary action onto the chromatographic strip (9) to dissolve the detectable agent present at the second end (11) of the chromatographic strip (9). In another alternative arrangement, the buffer pad (14) may be situated directly on top of the second end (11) of the chromatographic strip (9), such as on top of the detectable agents in the second end (11) of the chromatographic strip (9). Fluid applied onto the buffer pad (14) in this embodiment can dissolve the detectable agent and move the detectable agent from the second end (11) of the chromatographic strip in a second flow direction towards the first end (10) of the chromatographic strip (9). If the buffer pad (14) overlaps the chromatographic strip (9), the overlap may be in a range from about 0.5 mm to about 10 mm, typically from about 1 mm to about 8 mm, preferably from about 2 to about 5 mm, and more preferably about 2 to 3 mm.

The first absorbent pad (15) is situated at the first end (10) of the chromatographic strip (9) adjacent to the sample filter (12) on the side of the sample filter (12) away from the conjugate pad (13) or the buffer pad (14). An optional second absorbent pad (16) is in capillary contact with the first absorbent pad (15). In one embodiment, the optional second absorbent pad (16) is situated directly on top of the first absorbent pad (15). The first absorbent pad (15) overlaps the first end (10) of the chromatographic strip (9) by a distance sufficient to allow capillary flow of fluid from chromatographic strip (9) to the absorbent pad. This distance is in a range from about 0.5 mm to about 10 mm, typically from about 1 mm to about 8 mm, preferably from about 2 to about 5 mm, and more preferably about 2 to 3 mm. Additionally, a third absorbent pad can optionally be used.

A backing pad (17) can optionally be used to support the chromatographic strip (9), although certain chromatographic strips are available that already has a backing in place.

In one embodiment of the present invention, as shown in FIG. 3, at least one absorbent pad (15) is in capillary contact with the first sample filter (12). Optionally, a second absorbent pad (16) is situated on top of the first absorbent pad (15). The absorbent pads (15, 16), in one embodiment (FIG. 3), are situated on top of the first sample filter (12) but do not obstruct the application of sample or reagent onto the first sample filter (12). A third absorbent pad can optionally be used.

In as yet another embodiment of the present invention, as shown in FIG. 4, the absorbent pads (15, 16) are situated adjacent to the first sample filter (12) at the first end (10) of the chromatographic strip, on the side of the first sample filter (12) that is opposite the sample filter (18). As indicated above, the first sample filter (12) is typically a whole blood filter containing an agglutinating agent. However, as indicated below, when it is intended to apply buffer through Port 1, the first sample filter (12) can be replaced with a sample pad. This can be hydrophilic, as described above, and need not necessarily be pretreated with anti-erythrocyte antibody or agglutinating agent as described above.

The arrangement of FIG. 4 is particularly suited to the performance of sandwich immunoassays such as those for prostate specific antigen (PSA) or thyroid stimulating hormone (TSH).

Devices according to FIG. 3 or FIG. 4 can be operated in several modes. In one of those modes, sample (whole blood) is added to both Port 1 and Port 2. When whole blood as sample is added to both Port 1 and Port 2, both the first sample filter (12) and the second sample filter (18) are preferably whole blood filters to prevent blood cells, particularly erythrocytes, from entering the chromatographic strip. Alternatively, a buffer can be added to Port 1 and whole blood as sample is added to Port 2. In that alternative, the first sample filter (12) need not be a whole blood filter; that is, it need not contain an agglutinating agent. However, it is generally preferred that both the first sample filter (12) and the second sample filter (18) are whole blood filters, regardless of whether sample or buffer is to be added to Port 1 in the performance of the assay.

In the operation of an assay using the formats of FIG. 3 and FIG. 4, sample containing whole blood is added to both Port 1 (2) and Port 2 (3) of FIG. 1. In this format, both first sample filter (12) and second sample filter (18) are preferably blood filters to filter out RBCs. FIG. 7 illustrates the operation of a sandwich assay using the embodiment shown in FIG. 4. An aliquot of a sample containing RBC is applied to the first sample filter (12) at the first end (10) of the chromatographic strip (9). Fluid from the sample flows in the first lateral flow direction (21) from the first end (10) to the second end (11), flowing past the capture band (5) and the control bands (6 and 7). The analyte, if present, is captured at the capture band (5), the analyte-antibody forming a first immunocomplex at capture band (5). A second aliquot of the same sample is then applied to the second sample filter (18) at Port 2 (3). Fluid from the second sample filter (18) passes through a fluid collector (19 of FIG. 4) and a conjugate pad (13) to the second end (11), and then from the second end (11) in a second lateral flow direction (22) toward the first end (10), past the capture band (5) and the control bands (6 and 7). The analyte, if present is captured at the capture band (5) by the detection reagent, such as an antibody, the analyte-antibody complex forming a sandwich. In this format, the analyte, for example, HBsAg, applied to Port 2 (3) will first combine with the conjugate from the conjugate pad, for example, labeled anti-HBsAg antibody, such as anti-HBsAg antibody conjugated to colloidal gold, to form an analyte-conjugate complex, which then migrates to the capture zone and reacts with an anti-HBsAg antibody immobilized at the capture zone, which by that time would have also captured HBsAg from analyte applied to Port 1 (2).

In a variation, addition of sample to sample filter (12) at the first end (10) may be omitted and sample can be added directly to the second sample filter (18) at Port 2 (3). In this format, buffer is first added to Port 1 to prewet the test strip, the buffer flowing in a first lateral flow direction (21) from the first end (10) to the second end (11). Then sample is applied to Port 2 (3). Fluid from sample passes through the fluid collector (19) and a conjugate pad (13) to the second end (11) and from the second end (11) in a second lateral flow direction (22) past the capture band (5) and the control bands (6 and 7). The analyte in the sample combines with the conjugate to form a complex, the complex then flows to the capture band and the control bands. The analyte-conjugate complex is captured at the capture band (5), forming a sandwich.

Notably, when the strip configuration of FIG. 3 or FIG. 4 is used for determination of analytes in non-whole blood samples, such as serum or plasma samples, the sample filter (18) in FIG. 3, or the sample filters (12 and 18) in FIG. 4 does not contain an agglutinating agent. This configuration can be used for both sandwich assays and indirect assays.

Test strips according to the present invention can be configured to carry out assays that employ either unidirectional flow or bidirectional flow. As used herein, the term “unidirectional flow” is interpreted to mean that liquid is applied to the test strip at only one location, typically through Port-1. The term “unidirectional flow” includes formats such as “stop flow,” in which the liquid that is applied to the test strip at only one location stops flowing at a point located within the chromatographic medium, or “reversed flow,” in which the liquid that is applied to the test strip at only one location reverses its flow through the chromatographic medium during the performance of the assay. These operating formats can be arranged by one of ordinary skill in the art by suitable selection of the volume of fluid applied to the test strip and the size and absorptive capacities of absorbing elements of the test strip. In “bidirectional flow,” liquid is applied to the test strip at multiple locations, typically through both Port-1 and Port-2; and the liquid flows through sufficient capillary gradients in each of the directions (the first flow direction and the second flow direction).

The materials assembled for the present invention and the arrangements of the components of the test strip confer a unique advantage to the present invention, enabling the use of small volume of samples, efficient filtering of cells including red blood cells, efficient dissolution of the detectable agents and the achievement of consistent results in determination of presence and quantity of analytes.

For construction of the present test strip, the chromatographic strip (9) of the present invention can be composed of any suitable material that has a high protein binding capability and supports a lateral flow assay. Typically, the chromatographic strip (9) is a hydrophilic membrane and the protein binding is through noncovalent binding. Although Applicants do not intend to be bound by this theory, current theory of binding of proteins to nitrocellulose states that the initial interaction is electrostatic, but subsequently hydrophobic interactions and hydrogen bonds considerably strengthen the binding. An example of a chromatographic material is the commonly used nitrocellulose membrane, which has been treated to make it hydrophilic, such as one made by Millipore Corporation (Billerica, Mass.). Another example of a chromatographic membrane is one made up of particles of a polymer, such as polyethylene, fused together. Such particles can be spherical particles. An example of this type of membrane is the POREX Lateral-Flo membrane (POREX Corporation, Fairburn, Ga.). The chromatographic strip is of any size appropriate for the instrument or device used to read the results. For example, for use in conjunction with the device of U.S. Pat. No. 6,136,610, the chromatographic strip is about 5 mm×44 mm. When antigens, such as HIV antigen or HCV antigen, are coated on the chromatographic strip, they are preferably coated in a solution containing trehalose. A suitable concentration of trehalose in the solution is 1.0%. Other compounds are known that can stabilize immobilized antibodies, such as on nitrocellulose.

When antigens or antibodies are coated onto the chromatographic strip (9), due to its porous nature, the protein solution distributes itself throughout the depth of the nitrocellulose membrane. The proteins bind to the pore surfaces. Because of the method of application and the physics of the binding, more protein is bound to the top and center of the line compared to other areas wetted by the striping solution.

The chromatographic strip (9) of the present invention contains at least one capture band for capturing the analyte and at least one control band and, optionally, a second control band. When used in conjunction with the cassette of FIG. 1, the capture band, and the control band or bands can be viewed through the testing window (4). The capture band contains materials that are capable of capturing an analyte in a sample if the analyte is present. For example, if the lateral flow assay is intended to measure hepatitis B virus (“HBV”) surface antigen (HBsAg) in a blood sample, the capture band will contain antibody to HBsAg immobilized on the chromatographic strip at the capture band. One of the two controls typically is a high control (“HC”) and the other will be a low control (“LC”), as described above. In one embodiment of the invention, the chromatographic strip (9) will additionally contain conjugates or detectable agents at the second end (11) for detecting the captured analyte.

In the embodiment as exemplified in FIG. 2, the sample filter (12) is preferably a hydrophobic membrane, or alternatively a hydrophilic membrane or a synthetic composite of such as typically used in lateral flow assays for sample application. Examples of such sample filters include, but are not limited to hydrophobic filters such as glass fiber filters (Ahlstrom Filtration, Inc. Mount Holly Springs, Pa., USA), composite filters such as Cytosep (Ahlstrom Filtration or Pall Specialty Materials, Port Washington, N.Y.), and hydrophilic filters such as cellulose (Pall Specialty Materials). In one embodiment, a single sample filter is sufficient. In another embodiment, more than a single sample filter may be used. The present sample filter (12) does not require use of any nucleating agent or nucleating particles. However, it may contain an agglutinating agent, such as an antibody or a chemical compound, for example. The agglutinating agent may not be necessary when the assay is run as a bidirectional lateral flow assay when sample is added in Port-1 (2) and only in Port-1 (2), and a high concentration of a non-ionic detergent, such as TWEEN 20, is present in the conjugate release buffer for releasing or dissolving the conjugate. The concentration of the detergent in the conjugate release buffer is at least about 0.1%. The combination of the detergent and conjugate release buffer aids in washing the red blood cells or lysed red blood cells away from the capture and control bands, and decreasing the non-specific binding of analyte to sample filter. Sample filters (12, 18) in the devices of FIG. 3 and FIG. 4 are constructed similarly.

In the embodiments as exemplified in FIGS. 3 and 4, the second sample filter (18) is typically a hydrophobic membrane such as a glass fiber membrane (Ahlstrom Filtration, Inc.). In one aspect of this embodiment, the second sample filter (18) contains an agglutinating agent. In another aspect of the invention, the hydrophobic membrane is treated with a detergent, such as a non-ionic detergent, for example, TWEEN 20, at a concentration of about 0.002%, prior to addition of the agglutinating agent. The second sample filter (18) is available for application of a sample through Port-2 (3) of the cassette (1) of FIG. 1.

The appropriate size of the sample filter or filters (12, 18) can be as appropriate for the test strip within the parameters specified. For example, for use in conjunction with the device of U.S. Pat. No. 6,136,610, the sample filter is about 5 mm×8 mm. In one embodiment in which an agglutinating agent is present in the sample filter (12, 18), the sample filter is preferably pretreated with a detergent, such as a non-ionic detergent, for example, TWEEN 20, at a concentration of about 0.002%, prior to addition of the agglutinating agent for best results.

The agglutinating agent of the present invention typically includes an antibody directed to the cells or other materials to be filtered out. For example, if the materials to be filtered out are blood cells, the agglutinating agent of present invention includes an anti-red blood cell antibody and/or an anti-white blood cell antibody. The antibody can be directed to a cell surface antigen. For example, the anti-red blood cell (anti-RBC) antibody includes an anti-red blood cell membrane antibody such as anti-Band 3 antibody or anti-glycophorin antibody, such as anti-glycophorin A antibody. Such antibodies are commercially available, for example, rabbit anti-human RBC (Buo-shen Biotech, Xia-Men, China) or mouse anti-human RBC (Rui-Tai-En Scientific LLC, Anhui, China), at a concentration appropriate for the assay, such as in the range of about 0.1 mg/ml to about 1 mg/ml, typically 0.2 mg/ml to about 0.8 mg/ml, preferably 0.25 mg/ml to about 0.5 mg/ml.

In another embodiment of the present invention, the agglutinating agent is a chemical compound, such as a lectin. Lectins are proteins or glycoproteins that are capable of agglutinating cells and include, for example, concanavalin A, wheat germ agglutinin, and the agglutinins of Glycine max and Phaseolus vulgaris, abrin, soybean agglutinins and the like, either singly or in combination, as described in Goldstein et al. (1980). Nature 285: 66, and Schnebli, H. P. and Bachi, J. (1975), Reactions of lectins with human erythrocytes. Exot. Cell. Research. 91. Such agglutinins are also commercially available.

The conjugate pad (13) of the present invention is composed of a hydrophobic material, such as glass fiber (Pall Specialty Materials) and contains a conjugate or a detectable agent that can react with an analyte in a sample or with an analyte that is captured on the capture band on the chromatographic strip. The detectable agent includes, for example, antibodies or antigens specific for the analyte that are conjugated to a detectable material such as a colored material, a fluorescent material, or a chemiluminescent material. An example of a colored material is colloidal gold. The conjugate pad herein is of a size suitable for the chromatographic strip within the parameters described. For example, for use in conjunction with the device of U.S. Pat. No. 6,136,610, the conjugate pad is about 5 mm×8.5 mm. Typically, the conjugate pads are preblocked with a buffer solution containing trehalose and casein. Typically, the buffer solution contains from about 2.5% to about 7.5% trehalose. Preferably, the buffer solution contains about 5% trehalose. Typically, the buffer solution contains from about 0.25% casein to about 0.75% casein. Preferably, the buffer solution contains about 0.5% casein. More preferably, the buffer solution contains 5% trehalose and 0.5% casein. Typically, the conjugate is coated on the pads in a solution of 2.5% trehalose and 0.25% casein. The purpose of the trehalose is to stabilize the conjugate when dried on the conjugate pad, not to prevent binding to the conjugate pad or to the nitrocellulose. Prevention of binding is typically done with a blocking protein. A suitable blocking protein is 0.5% Hammarsten casein that is base solubilized. Prevention of binding can also be accomplished by using glass fiber that has been processed with a synthetic polymer binder. Other agents are known which stabilize conjugates when dried on conjugate pads and the invention is not limited to the use of conjugate pads preblocked with trehalose and casein.

The buffer pad (14) of present invention is a hydrophilic membrane or a synthetic composite, such as a Cytosep membrane (Ahlstrom Filtration, Inc.). In the embodiment exemplified in FIG. 2, the buffer pad (14) is accessible in the cassette (1) for application of reagents at Port-2 (3). The buffer pad (14) herein is of a size suitable for the chromatographic strip within the parameters described. For example, for use in conjunction with the device of U.S. Pat. No. 6,136,610, the buffer pad is about 5 mm×13 mm.

The absorbent pad (15, 16) of the present invention is a hydrophilic membrane that can absorb liquid, such as cellulose (Whatman, Kent, U.K) or a cellulose-glass fiber composite (Whatman, Kent, UK). The absorbent pad herein is of a size suitable for the chromatographic strip within the parameters described. For example, for use in conjunction with the device of U.S. Pat. No. 6,136,610, the absorbent pad is about 5 mm×27 mm.

The backing pad (17) of the present invention may be made of any inert material that is capable of supporting the chromatographic strip, such as a piece of plastic material (G&L Precision Cutting, San Jose, Calif.). The size of the backing pad (17) is as suitable for the chromatographic strip within the parameters described. For example, for use in conjunction with the device of U.S. Pat. No. 6,136,610, the backing pad is about 5 mm×60 mm.

The fluid collector (19) of the present invention is a hydrophobic membrane, just like the hydrophobic membrane of the conjugate pad (13). Unlike the conjugate pad (13), the fluid collector (19) does not contain any detectable agents. The size of the fluid collector (19) is as suitable for the chromatographic strip within the parameters described. For example, for use in conjunction with the device of U.S. Pat. No. 6,136,610, the fluid collector is about 5 mm×13 mm.

Alternatively, when the first sample filter (12) is replaced with a sample pad, the sample pad is a hydrophilic membrane such as Cytosep (Ahlstrom Filtration, Inc.) in one embodiment of the invention, such as shown in FIG. 3, where the sample pad is available for application of a reagent, such as a buffer, through Port-1 (2) of the cassette (1) of FIG. 1. The sample pad is useful, for example, for application of a buffer to pre-wet the chromatographic strip (9) prior to addition of a sample to the sample filter (18). The size of the sample pad is as suitable for the chromatographic strip within the parameters described. For example, for use in conjunction with the device of U.S. Pat. No. 6,136,610, the sample pad is about 5 mm×18 mm. The sample pad can be optionally pretreated with an anti-erythrocyte antibody or other agglutinating agent, but need not be if buffer is to be applied to Port-1.

In another embodiment of the invention, such as exemplified in FIG. 4, when the first sample filter (12) is replaced with a sample pad, the sample pad can be a hydrophobic membrane, just like the hydrophobic membrane of the sample filter (18). In this embodiment, the sample pad may also contain an agglutinin. Alternatively, the sample pad can be hydrophilic, with or without an agglutinin, as described above.

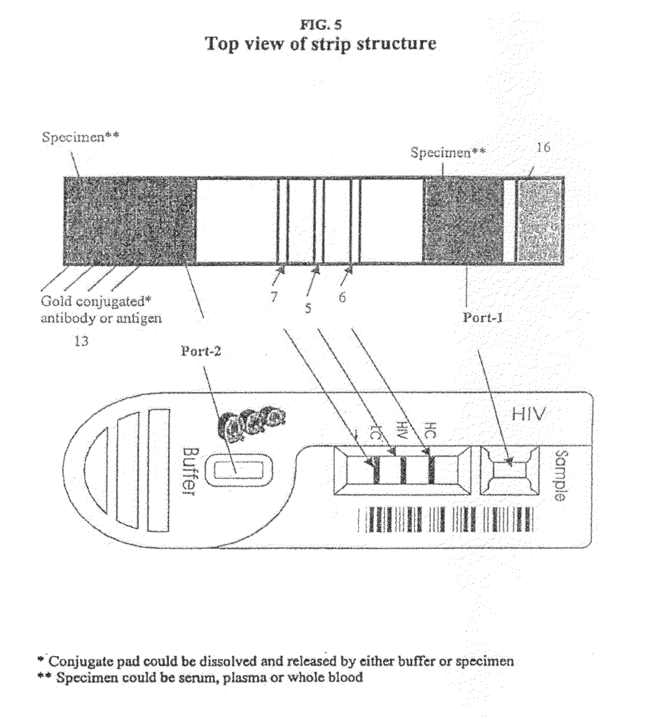

Referring to FIG. 5, FIG. 5 shows the correspondence between the top plan view of the test strip of the present invention and the cassette that may be used therewith. In this embodiment, the detectable agents may be incorporated into the chromatographic strip at the second end (11) or may be present in a conjugate pad (13). When this strip configuration is used for a sandwich assay, a sample to be analyzed may be added to both Port-1 (2) and Port-2 (3); or alternatively, a sample may be added to Port-2 (3), but a reagent such as a buffer instead of a sample may be added to Port-1 (2). When it is used for an indirect assay, a sample may be added to Port-1 (2), a buffer may be added to Port-2 (3).

While the present invention provides advantages such as the efficient separation of red blood cells from the fluid in a sample and lack of dependency on cell volume, the present invention can also be used for determination and quantitation of one or more analytes in samples in which no cells are present or in which the cells present are not red blood cells. Hence, the samples to be tested include serum, plasma and whole blood.

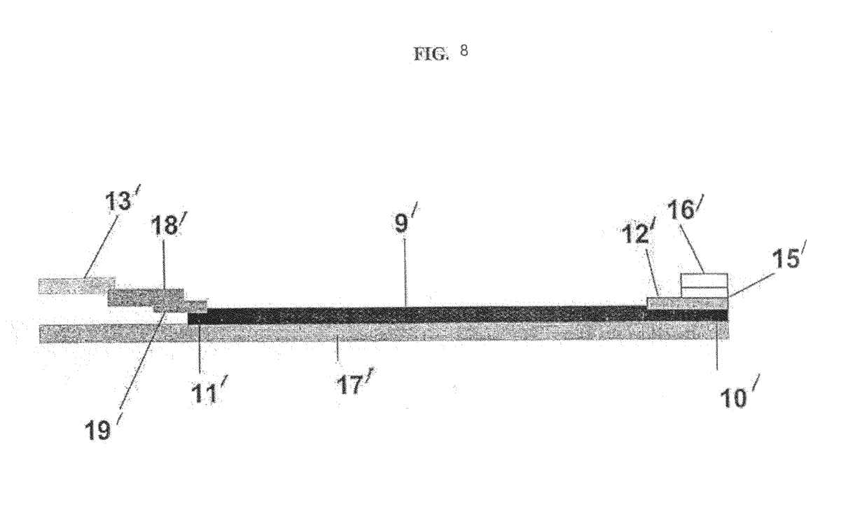

Another embodiment of a test strip according to the present invention is shown generally in FIG. 8. In general, FIG. 8 is a variation of the test strip according to the present invention shown in FIG. 3 but one in which the sample reacts with conjugate before reaching the sample filter, at least for sample applied to the conjugate pad, typically through Port-2. In this embodiment, the chromatographic strip (9′) has a first end (10′) and a second end (11′), a sample filter (18′), a fluid collector (19′), and a conjugate pad (13′), all situated at the second end (11′) of the chromatographic strip (9′), together with a first sample filter (12′), at least one absorbent pad (15′), and optionally a second absorbent pad (16′) that is in capillary contact with the first absorbent pad (15′), all situated at the first end (10′) of the chromatographic strip (9′). Additionally, a third absorbent pad can optionally be used. The test strip of FIG. 8 has capture and control bands as in FIG. 3 (not shown). Sample can be applied to the conjugate pad (13′) as well as to the first sample filter (12′). The sample filter (18′) and the fluid collector (19′) can be constructed of the same macroporous material, but this is not required. It is preferred that the sample filter (18′) have the same pore size as the conjugate pad (13′), and that the fluid collector (19′) have a smaller pore size. The capillary gradient is therefore (19′)>(18′)>(13′) because of the contact with (9′). In operation of the device of FIG. 8 when the device is used to perform a bidirectional assay, sample is applied to both the conjugate pad (13′) as well as to the first sample filter (12′); the first sample filter (12′) is typically accessed through Port-1 and the conjugate pad (13′) is typically accessed through Port-2.

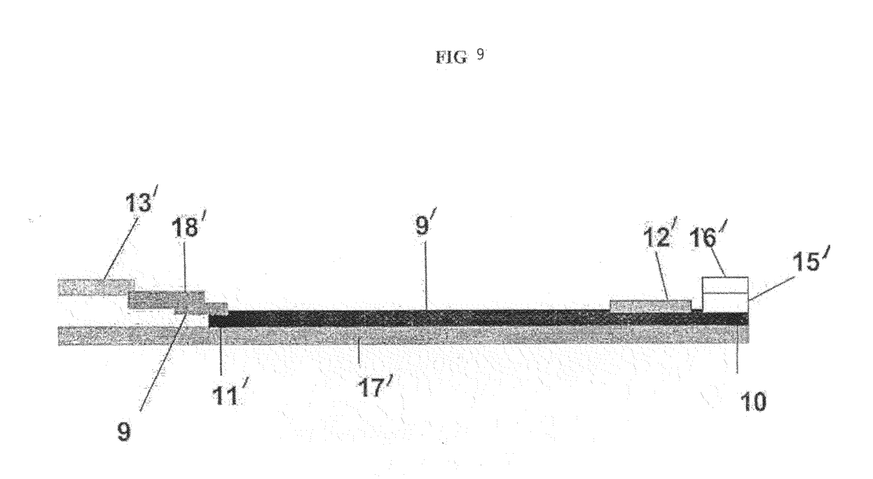

Still another embodiment of a test strip according to the present invention is shown generally in FIG. 9. In general, FIG. 9 is a variation of the test strip according to the present invention shown in FIG. 4 but one in which the sample reacts with conjugate before reaching the sample filter, at least for sample applied to the conjugate pad, typically through Port-2, as shown above for FIG. 8. The test strip of FIG. 9 has capture and control bands as in FIG. 4 (not shown). The test strip of FIG. 9 is similar to that of FIG. 8 except that the sample pad (12′) is in direct contact with the chromatographic strip (9′) and is located further away from the first end (10′) of the chromatographic strip (9′) than are the absorbers (15′) and (16′). By contrast, in the test strip of FIG. 8, the absorbers (15′) and (16′) are stacked atop the sample pad (12′) such that the surface of the sample pad (12′) is partially covered by the absorbers (15′) and (16′). Additionally, a third absorbent pad can optionally be used. Sample can be applied to the conjugate pad (13′) as well as to the first sample filter (12′). The sample filter (18′) and the fluid collector (19′) can be constructed of the same macroporous material, but this is not required. It is preferred that the sample filter (18′) have the same pore size as the conjugate pad (13′), and that the fluid collector (19′) have a smaller pore size. The capillary gradient is therefore (19′)>(18′)>(13′) because of the contact with (9′). In operation of the device of FIG. 9 when the device is used to perform a bidirectional assay, sample is applied to both the conjugate pad (13′) as well as to the first sample filter (12′); the first sample filter (12′) is typically accessed through Port-1 and the conjugate pad (13′) is typically accessed through Port-2.

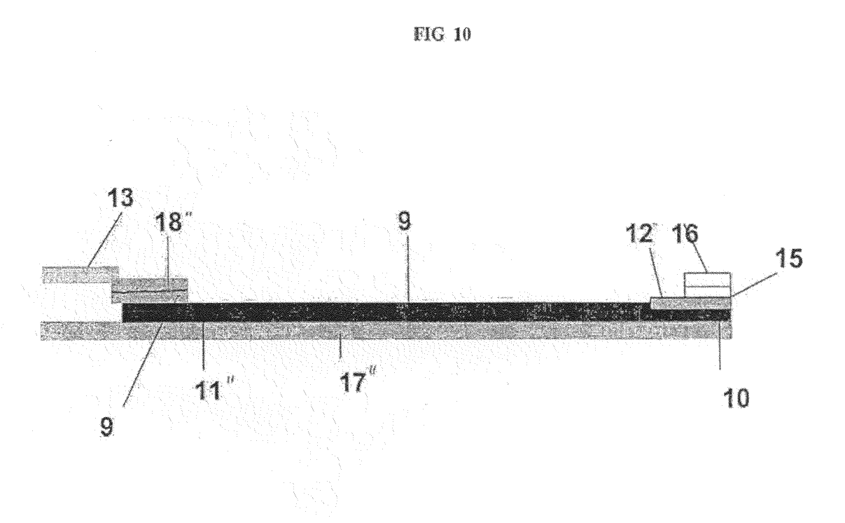

Yet another embodiment of a test strip according to the present invention is shown generally in FIG. 10. In general, FIG. 10 is a variation of the test strip according to the present invention shown in FIG. 3 but one in which, stacked atop the second end of the chromatographic strip, are, in order, a fluid collector, a sample filter, and a conjugate pad. Typically, the fluid collector and the sample filter are in line, and the conjugate pad is offset so that it partially overlaps the sample filter. In this embodiment, the chromatographic strip (9″) has a first end (10″) and a second end (11″), a sample filter (18″), optionally, a fluid collector (19″), and a conjugate pad (13″), all situated at the second end (11″) of the chromatographic strip (9″), together with a buffer pad (12″), at least one absorbent pad (15″), and optionally a second absorbent pad (16″) that is in capillary contact with the first absorbent pad (15″), all situated at the first end (10″) of the chromatographic strip (9″). Additionally, a third absorbent pad can optionally be used. The test strip of FIG. 9 has capture and control bands as in FIG. 3 (not shown). Sample can be applied to the sample filter (18″). The sample filter (18″) and the fluid collector (19″) can be constructed of the same macroporous material, but this is not required. It is preferred that the sample filter (18″) have a pore size smaller than the conjugate pad (13″), and that the fluid collector (19″) have a smaller pore size than the sample filter (18″). The capillary gradient is therefore (11″)>(18″,19″)>(13″) because of the contact with (9″). In operation, the following sequence is followed: (1) optionally prewet the chromatographic strip (9″) from the buffer pad (12″) at the first end; (2) add sample to the sample filter (18″), allow the fluid to flow onto the optional fluid collector (19″) or directly onto the chromatographic strip (9″) in a direction towards the second end (11″), past the capture and control bands, and allow the analyte to be captured at the capture band, if present; (3) add buffer to conjugate pad (13″) to release the conjugate, allow the conjugate to move past the sample filter (18″), towards the second end (11″), past the capture and control bands, and allow the mobilizable detectable agent to detect captured analyte at the capture band.

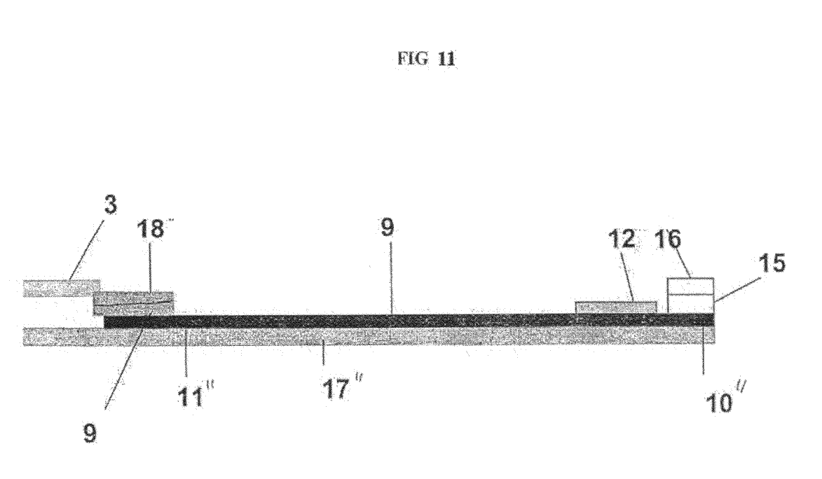

Similarly, FIG. 11 shows an embodiment generally similar to that of FIG. 10 except that the relationship of the buffer pad (12″), the absorbent pads (15″, 16″) and the first end (10″) of the chromatographic strip (9″) is as shown in FIG. 4 or FIG. 9. The operation of the device of FIG. 11 is substantially similar to that of FIG. 10.

Accordingly, another embodiment of a test strip according to the present invention generally is a test strip for a lateral flow assay for detection of at least one analyte in a sample, comprising:

(1) a chromatographic strip comprising a first end and a second end, at least one capture band comprising an immobilized capture agent for capturing the at least one analyte, and at least one control band comprising an immobilized control agent for determination of non-specific binding;

(2) a conjugate pad, wherein the conjugate pad is in capillary contact with the second end of the chromatograph strip, and wherein the conjugate pad comprises a mobilizable detectable agent that is capable of binding to the at least one analyte or to the capture agent after capturing the analyte;

(3) a sample filter that is adjacent to the conjugate pad on the side closer to the second end, wherein the sample filter optionally comprises an agglutinating agent, and the sample filter is in capillary contact with the chromatographic strip;

(4) optionally a fluid collector that, if present, is situated between the sample filter and the chromatographic strip;

(5) optionally, a buffer pad situated at the first end of the chromatographic strip and is in capillary contact with the chromatographic strip;

(6) a first absorbent pad situated at the first end of the chromatographic strip that is in capillary contact with the chromatographic strip, either directly or indirectly; and

(7) optionally, a second absorbent pad that, if present, is in capillary contact with the first absorbent pad; wherein the test strip allows detection or quantitation of an analyte in a sample containing whole cells.

The method of conducting a lateral flow assay using the test strip of the present invention can be illustrated by referring to FIG. 6. FIG. 6 shows the top plan view of one embodiment of the present invention, showing bi-directional lateral flow of sample and reagents in an assay, such as in the embodiment of FIG. 2. In this embodiment, a sample is applied onto a sample filter (12). Fluid from the sample filter (12) migrates to the chromatographic strip (9) at the first end (10) and flows in a first flow direction (21) past the capture band (5) and the control bands (6, 7) towards the second end (11) of the chromatographic strip (9). Between the control band (7) and the conjugate pad (13), fluid from the sample ceases flow in the first flow direction (21); if there is excessive fluid, after the second liquid is applied onto Port-2 (3), the excessive fluid in the first flow will reverse and flow in the second flow direction (22) back towards the first end (10) of the chromatographic strip (9). The analyte in the sample, if present, is captured primarily at the capture band (5) during the course of the fluid flow in the first flow direction (21), and secondarily during the course of fluid flow in the second flow direction (22). The bidirectional lateral flow aids pre-wet the chromatography strip, so that the chemicals on its surface could be dissolved and distributed evenly before labeled reagents flowed into this area; it also aids in washing contaminants away from the capture and control bands, reducing background noise in the assay. A reagent, such as a buffer or conjugate release buffer suitable for the assay, is applied to the buffer pad (14) in Port-2, in an amount sufficient to dissolve or release the conjugate. A particularly suitable conjugate release buffer is 1×PBS containing 0.1% Tween20, 0.01% casein, 0.3% SDS, 0.2 mM EDTA and 0.1% sodium azide. The released conjugate migrates from the second end (11) of the chromatographic strip (9) in a second flow direction (22) towards the first end (10) of the chromatographic strip (9) and interacts with the analyte at the capture band (5). The conjugate is made relevant to the analyte to be tested. For example, for detection of human antibodies in a human blood sample, the conjugate can be an anti-human IgG, (or IgM when it is an human IgM testing), such as, but not limited to, goat anti-human IgG, rabbit anti-human IgG, or murine anti-human IgG conjugated to colloidal gold.

In the absence of an agglutinin in the sample filter (12) in the conduct of a bidirectional lateral flow assay, red blood cells present in a sample may leak onto the chromatographic strip (9) creating high background noise and therefore a reduced signal-to-noise ratio. The inventors herein have discovered that this background problem may be reduced significantly if a detergent, such as a non-ionic detergent, for example, TWEEN 20, is present in the conjugate release buffer, at a relatively high concentration, for example, at least about 0.1%. The combination of the detergent and conjugate release buffer aids in washing the red blood cells or lysed red blood cells away from the capture and control bands, and decreasing the non-specific binding of analyte to sample filter.

If an agglutinin is used in the sample filter (12), such as an anti-red blood cell antibody, to remove red blood cells, then the sample filter (12) is pretreated with a detergent, such as a non-ionic detergent, such as TWEEN 20. A low concentration of the detergent is used for this purpose, such as, for example, about 0.002%. The application of non-ionic detergent aids to change the hydrophobic surface of sample filter to slightly hydrophilic, so that the agglutinin agent could bind to sample filter more sufficiently.

FIG. 7 illustrates the conduct of a sandwich assay of an embodiment of the present invention as shown in FIG. 4. An aliquot of a sample such as one containing red blood cells is applied to the first sample filter (12) at the first end (10) of the chromatographic strip (9). Fluid from this aliquot flows in the first flow direction (21) from the first end (10) of the chromatographic strip (9) towards the second end (11), flowing past the capture band (5) and the control bands (6, 7). The analyte, if present, is captured at the capture band (5). A second aliquot of the same sample is then applied to the second sample filter (18) at Port-2 (3) the second end (11) of the chromatographic strip (9). Fluid in the second sample filter (18) passes through a fluid collector (not shown) and a conjugate pad (13) to the second end (11) of the chromatographic strip (9), which then flows in the second flow direction (22) from the second end of the strip towards the first, flowing past the capture band (5) and control bands (6, 7). The analyte, if present, is also captured at the capture band (5), where the analyte together with the detection reagent at the capture band form a sandwich.

Alternatively, initial addition of sample to the sample pad at the first end (10) of the chromatographic strip (9) may be omitted and instead sample added directly to the second sample filter (18) at Port-2 (3) at the second end (11) of the chromatographic strip (9). Fluid in the sample filter (18) passes through a fluid collector (not shown) and a conjugate pad (13) to the second end (11) of the chromatographic strip (9), which then flows in the second flow direction from the second end of the strip towards the first, flowing past the capture band (5) and control bands (6, 7). The analyte, if present, is also captured at the capture band (5), where the analyte together with the detection reagent at the capture band form a sandwich. Typically, in this mode of operation, buffer is added to the first sample filter (12) so that the buffer flows in the first flow direction from the first end of the strip toward the second to prewet the strip.

Similar sequences of operation can be carried out with the device shown in FIG. 3.

As indicated above, test strips according to the present invention can be used to detect multiple analytes in a single assay. For example, the sample can contains two analytes and the chromatographic strip can then comprise two separate capture bands, each capture band comprising an immobilized capture agent that is specific for capturing one analyte but not the other. Alternatively, the sample can contain three analytes and the chromatographic strip can then comprise three separate capture bands, each capture band comprising an immobilized capture agent that is specific for capturing one analyte but not the other two. Those of ordinary skill in the art can select appropriate capture agents for combinations of analytes desired to be assayed in a single assay according to the nature of the analytes and the specificities of the capture agents, such as antibodies, for them.

The amount of analyte captured at the capture band can be quantitated as described in U.S. Pat. No. 6,136,610. However, other methods of quantitation are possible. Test strips according to the present invention can also be used for qualitative or semiquantitative determinations.

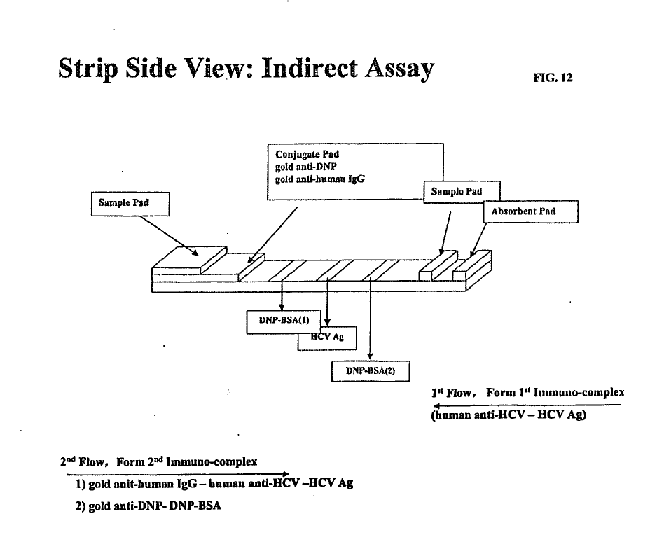

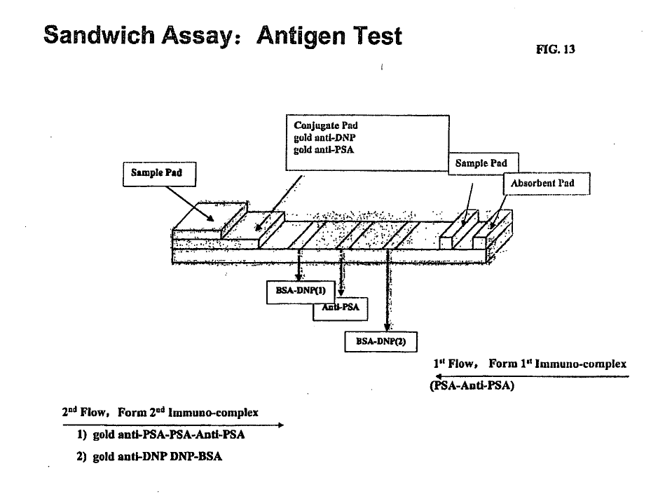

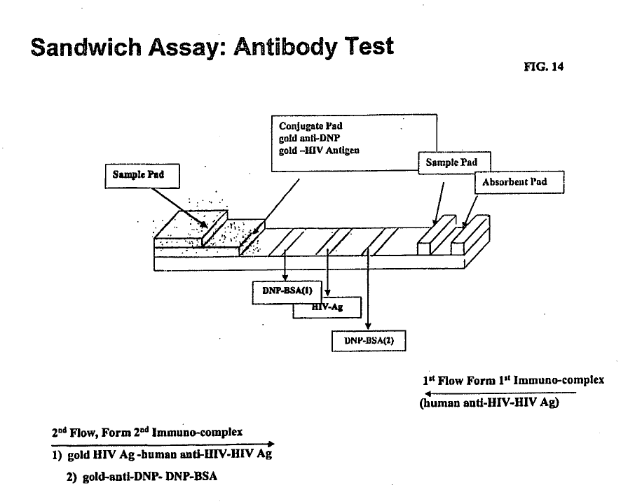

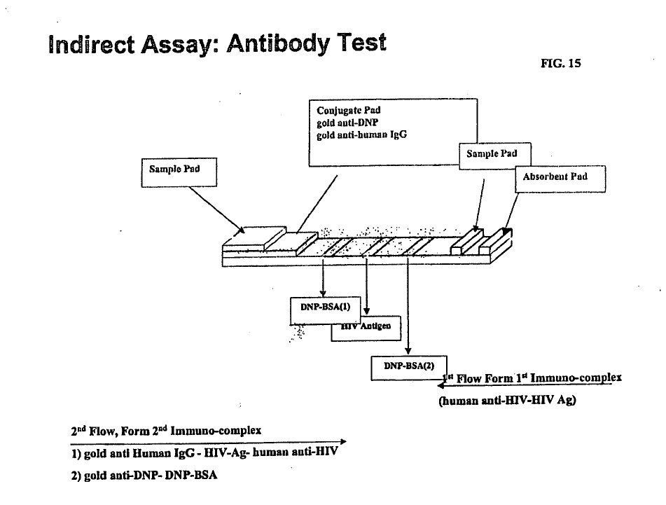

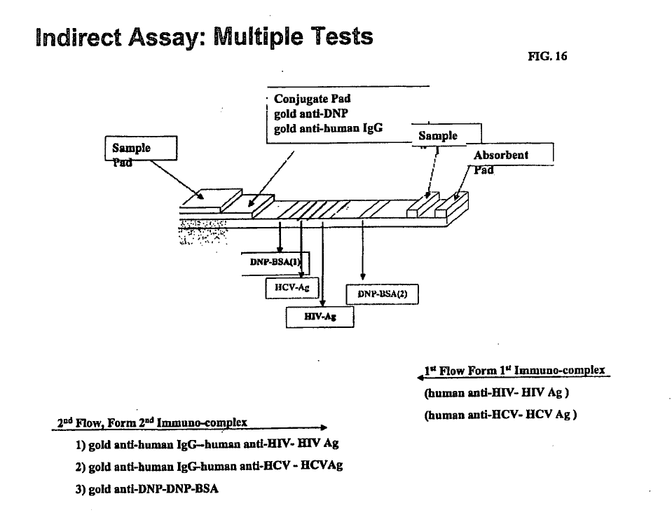

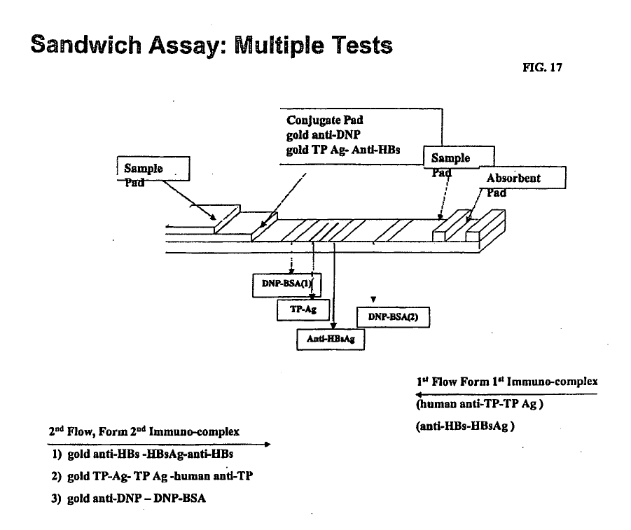

Specific embodiments of test strips according to the present invention are illustrated in FIGS. 12-17. The devices of FIGS. 12-17 all use gold anti-DNP antibody and DNP-BSA as a control and operate in a bidirectional mode. FIG. 12 is a detailed side view of an embodiment of a test strip capable of performing an indirect assay for human hepatitis C virus (HCV). FIG. 13 is a detailed side view of an embodiment of a test strip capable of performing a sandwich assay for prostate specific antigen (PSA). FIG. 14 is a detailed side view of an embodiment of a test strip capable of performing a sandwich assay for antibody specific for human HIV. FIG. 15 is a detailed side view of an embodiment of a test strip capable of performing an indirect assay for antibody specific for human HIV. FIG. 16 is a detailed side view of an embodiment of a test strip capable of performing an indirect assay for antibody specific for human HIV and for antibody specific for HCV in the same sample. FIG. 17 is a detailed side view of an embodiment of a test strip capable of performing a sandwich assay for hepatitis B surface antigen (HBsAg) and for Treponema pallidum antigen in the same sample.

The components used herein including the absorbent pad, the sample filter, the buffer pad, the chromatographic strip, and the conjugate pad have the properties set forth in Table 1, as specified by the manufacturer thereof. However, other alternative components can be used and are known in the art.

Suitable analytes include, but are not limited to antigens, antibodies, hormones, drugs, cell proteins, DNAs, cardiac markers, tumor or cancer markers, autoimmune disease markers, or any macromolecule that could raise antibodies. When the analyte is an antigen, the antigen can be an antigen associated with an infectious agent. The infectious agent can be a virus, a bacterium, a fungus, or a prion. When the infectious agent is a virus, the virus can be selected from the group consisting of HIV, hepatitis virus A, B, C, and D, herpes simplex virus, cytomegalovirus, papilloma virus, Ebola virus, SARS virus, Rhinovirus, and Vaccinia virus, but is not limited to those viruses. When the infectious agent is a bacterium, the bacterium can be a Gram-positive bacterium or a Gram-negative bacterium. The bacterium can be selected from the group consisting of Bacillus anthracis, Escherichia coli, Helicobacter pylori, Neisseria gonorrheae, Salmonella species, and Shigella species, but is not limited to those bacteria. When the infectious agent is a fungus, the fungus can be a Mycosporum species or an Aspergillus species, but is not limited to those fungi.

When the analyte is a hormone, typically it is selected from the group consisting of hCG, thyroxin, TSH, glucagons, insulin, relaxin, prolactin, luteinizing hormone, melanotropin, somatotropin, follicle-stimulating hormone, gastrin, bradykinin, vasopressin, and other releasing factors; however, other hormones of physiological or pathological interest can be the analyte.

When the analyte is a cancer or tumor marker, typically it is selected from the group consisting of prostate specific antigen (PSA), carcinoembryonic antigen (CEA), and α-fetoprotein; however, other cancer or tumor markers can be the analyte.

When the analyte is a cardiac marker, the cardiac marker is typically selected from the group consisting of Troponin-I, Troponin T, Creatine kinase-MB isoforms (CK-MB), myoglobin, C-reactive protein (CRP), fatty acid binding protein (FABP), glycogen phosphorylase isoenzyme BB (GPBB), B-type natriuretic peptide (BNP) and pro-BNP; however, the analyte can be another cardiac marker.

Still other analytes can be assayed by test strips and methods according to the present invention. For example, tissue-specific cell surface markers can be assayed. Separation of cell populations based on these markers has been performed using lectins (Reisner and Sharon, Trends in Biochem Sci (TIBS) 29, 1980), blood leukocyte surface glycoproteins (Gahmberg and Anderssen, N Y A S (1978) 312, in Fibroblast Surface Proteins eds. Vahery, Ruslahti and Mosher), estrogen steroid receptors (Thompson, Cancer Treatment Reports (1978) 63(2) 180, erythrocyte insulin receptors (Bhathena et al, Horm Metab Res (1981) 13:179), or multiple markers as in the case of lymphocytes. Further separation of subpopulations is possible based on markers identified with specific cell functions as in the case of the T lymphocytes (Reinberg and Schlossman, N Eng J Med (1980) 303:1153).

Similarly, tissue-shared cell surface markers can be assayed. Some cell surface markers are present on multiple cell types. An example of these are the Major Histocompatibility Complex Human Lymphocyte Antigen (HLA) system, LETS protein, p glycoprotein (Kartner et al, Science (1983) 221:1285) and transferrin receptors (Omary et al, Nature (London) (1980) 286:888).

Other analytes include viral-associated cell surface markers. Cell membrane antigens can also result from viral infection. The mumps H—N glycoprotein detectable by RIA, immunofluorescence and hemagglutination inhibition represents a viral marker on infected cells (Sever et al, Infect & Immun (1982) 35(1):179). Similarly, markers resulting from Herpes Simplex 1 and 2 infection are recognizable on the host cell surface by immunofluorescence (Stewart and Herrmann, “Herpes Simplex Virus” in Manual of Clinical Immunology, 2nd edition, edited by N. R. Rose and H. Friedman, American Society for Microbiology, Washington, D.C., 1980).

Still other analytes include tumor-specific cell surface markers. Neoplastic and oncogenic transformation results in the alteration of the cell phenotype as expressed in cell surface proteins. These can be observed as variations in the presence of cell surface antigens normally expressed on the cell surface, appearance of “altered self antigens,” appearance of embryonic cell surface antigens and the presence of tumor specific molecules. Felsted et al (Canc Res (1983) 43:2754) have described cell membrane changes during the differentiation of promyelocytic leukemia cells. Neoplastic transformation induced changes in cell phenotype are presented in a review by Poste (in Cancer Invasion and Metastasis: Biologic Mechanisms and Therapy edited by S. B. Day et al, Raven Press, New York, 1977). Similar review articles describe phenotypes of leukemic cells (Greaves et al in Proc of International Symposium on Human Lymphocyte Differentiation: Its Application to Cancer, edited by Seron and Rosenfeld, North Holland Publishing, Amsterdam, 1978), B Lymphocytes (Thorsky et al, IBID), and Acute Lymphocytic Leukemia Cells (Greaves et al, Science 234, 1986). The identification of tumor specific antigens or markers and their association with tumors of specific tissue types permits clearer diagnosis and subsequent monitoring during therapy. A number of tumor surface proteins have been identified. Several examples include: a mutated rat gene p21 tumor lymphocyte protein (Bos et al, Nature (London) (1985) 315:726, and Clark et al, PNAS (USA) (1985) 82:5280); an Acute Lymphocyte Leukemia (ALL) Associated antigen GP 100 Ph1 (Greaves et al, Blood (1983) 61:628); Human T cell Leukemia Associated Antigen (HTLA) (Seon et al, J of Immunol (1981) 127(6):2580); a Human Lung Tumor Associated Antigen (Braatz et al, J Nat Cancer Inst (1978) 61(4):1035), an estrogen 24,000 MW Human breast cancer marker (Adams et al, Cancer Res (1983) 43:4297); a Human Leiomyosarcoma antigen (Deng et al, Lancet, Feb. 21, 1981, p. 403); and a Human Mammary carcinoma antigen (Schlom et al, PNAS (1980) 77 (11):6841). Further concerning tumor markers, the concept of “altered self antigens” proposed by Edelman, Science (1976) 197:218 describes the presence of modified cell surface antigens normally indigenous to a cell type which are altered due to neoplastic transformation. These aberrant cells are viewed by the immune surveillance system as abnormal and they are capable of eliciting an immune response (Burnet, Brit Med J (1957) 1:179, and Nature (1970) 226:123). The reappearance of embryonic antigens has also been observed following the neoplastic transformation of cells. Carcinoembryonic antigen (CEA), Fetal Embryonic antigen (FEA) and Tumor Specific Transplantation Antigens (TSTA) have been useful in the serodiagnostic detection of carcinomas and sarcomas (Mitchison, “Immune Surveillance” in B and T Cells in Immune Recognition edited by F. Loors and G. E. Roelants, Wiley and Sons, New York, 1977).

Other analytes include lipoproteins, enzymes, immunoglobulins, lymphokines, cytokines, and drugs, including any drug to which antibodies can be prepared through the process of haptenization. In haptenization, a molecule that is too small to elicit antibody formation when injected by itself into an antibody-forming animal can be coupled to a larger carrier molecule, such as a protein molecule such as keyhole limpet hemocyanin, and injected in that form to form antibodies.

Other protein analytes include transcortin, erythropoietin, transferrin, various globulins, thyroxin-binding globulin, the immunoglobulins of various subclasses A, G, D, E, and M, various complement factors, blood clotting factors such as fibrinogen, Factor VIII, tissue thromboplastin, and thrombin.

Still other analytes include drugs, both therapeutic drugs and drugs of abuse or having a potential for abuse. Many drugs that can serve as analytes are disclosed in U.S. Pat. No. 3,996,345 to Ullman et al., incorporated herein by this reference. These drugs include, but are not limited to, alkaloids and metabolites of alkaloids, including morphine, cocaine, mescaline, and lysergic acid, as well as synthetic opiates. Still other drugs include methadone, meperidine, amphetamine, methamphetamine, glutethimide, diphenylhydantoin, and drugs which come within the category of benzdiazocycloheptanes, phenothiazines and barbiturates. Still other drugs include epinephrine, ephedrine, L-dopa, and norepinephrine. Still other drugs include the tranquilizer Meprobamate, Tergitol and succinimides, such as Ethoxsumide. Still other drugs include tetrahydrocannabinol, cannabinol, and derivatives thereof, primarily compounds derived from marijuana, synthetic modifications and metabolites thereof. Still other drugs include steroids such as estrogens, gestogens, androgens, adrenocortical hormones, bile acids, cardiotonic glycoids, aglycones, saponins; and sapogenins. Typically, small molecules such as steroids, alkaloids, and peptides require haptenization as discussed above for the production of antibodies.

Although the foregoing discussion has focused on substances that can be determined by antigen-antibody interactions as analytes, the use of the term “analyte” is not to be taken to limit the scope of substances that can be assayed with devices and methods according to the present invention to substances that can be determined by antigen-antibody interactions. For example, for analytes for which a specific binding protein of sufficiently great specificity exists, either the antibody that is immobilized to the chromatographic strip or the antibody that is labeled with the conjugate can be replaced with a suitable specific binding protein. These include, but are not limited to, intrinsic factor protein as a member of a specific binding pair for the determination of Vitamin B₁₂, the use of folate-binding protein to determine folic acid, the use of a lectin as a member of a specific binding pair for the determination of a carbohydrate, or the use of a cytokine, lymphokine, or growth factor receptor such as interleukin-1 receptor to determine the corresponding cytokine, lymphokine, or growth factor.

Additionally, the term “analyte” can encompass nucleic acids such as DNA or RNA as long as suitable specific binding macromolecules exist for these nucleic acids. These suitable specific binding macromolecules can be proteins that bind to nucleic acids in a sequence-specific manner, or can be nucleic acid molecules or nucleic acid molecule analogues that bind to the sequence to be detected according to the Watson-Crick base pairing rules. If the nucleic acid to be detected is of sufficient length, the nucleic acid to be detected can hybridize at one sequence within the nucleic acid molecule to an immobilized complementary nucleic acid, and can then hybridize at another sequence within the nucleic acid molecule with a labeled nucleic acid, a process generally referred to as “sandwich hybridization,” and described in greater detail in, for example, U.S. Pat. No. 6,825,331 to Manoharan et al., incorporated herein by this reference.

When the sample is applied to Port-1 in the performance of a bidirectional assay and a buffer is applied to Port-2 for flow in the second direction, a suitable buffer is one that is compatible in pH and ionic strength with the sample and any reagents added to the sample. The buffer should not interact with any analytes or other macromolecules in the sample. Suitable buffers include, but are not limited to, phosphate buffered saline, Ringer's solution, Hank's solution, and buffered solutions buffered with (tris)hydroxymethylaminomethane (Tris™). The same types of buffers can be used when the buffer is applied to Port-1 to prewet the chromatographic strip and a sample is applied to Port-2.

TABLE 1
Summary of Membrane Selection in Port-2

| | | | | | | | Ability to |
|---|---|---|---|---|---|---|---|
| | | | Pore | | Capillary | | filter red |
| | Chemical | Hydrophobic/ | Size | Flow Rate | rise | Lower | blood cells |
| Top Layer | Nature | Hydrophilic | (μm) | (ml/min) | (mm/min) | Layer | (Yes/No) |
| #111 | Cellulose | Hydrophilic | 1 | 130 | 51 | Conjugate | No |
| | | | | | | Pad | |
| #141 | Glass | Hydrophobic | 3 | 350 | 79 | Conjugate | No |
| | fiber | | | | | Pad | |
| #142 | Glass | Hydrophobic | 6 | 300 | 55 | #142 | No |
| | fiber | | | | | | |
| #142 | Glass | Hydrophobic | 6 | 300 | 55 | Conjugate | Yes |
| | fiber | | | | | Pad | |
| #1660 | CytoSep | Hydrophilic | 3 | 100 | 52 | Conjugate | No |
| | | | | | | Pad | |
| #1661 | CytoSep | Hydrophilic | 5 | 260 | 41 | Conjugate | No |
| | | | | | | Pad | |
| #1662 | CytoSep | Hydrophilic | 3 | 35 | 46 | Conjugate | No |
| | | | | | | Pad | |
| #1663 | CytoSep | Hydrophilic | 2 | 35 | 46 | Conjugate | No |
| | | | | | | Pad | |
| #319 | CytoSep | Hydrophilic | 19 | 375 | 54 | Conjugate | No |
| | | | | | | Pad | |
| Conjugate | Glass | Hydrophobic | 42 | 250 | 46 | Conjugate | No |
| Pad | fiber | | | | | Pad | |

### INDUSTRIAL APPLICABILITY

The present invention may be advantageously employed in diagnostic settings including point of care settings, such as in a doctor's office or clinic or in a battlefield, for determining presence and quantity of analytes present in samples that may or may not contains cells, such as red blood cells, white blood cells and other cell types. The materials and methods of the present invention are useful, for example, in the detection of disease agents or antibodies thereto, including HIV, HAV, HBV, HCV, HSV, HPV, CMV, SARS virus, vaccinia virus, as well as other molecules, including, for example, deoxypyrodinoline (a bone resorption marker), human serum albumin, drugs of abuse, protein markers such as prostate specific antigen (“PSA”), kidney disease proteins such as lactate dehydrogenase, N-acetyl-β-D-glucosamine, pregnancy or fertility associated hormones such as chorionic gonadotropin (“hCG”) and markers of urinary tract infection. The determination of blood borne analytes, such as therapeutic drugs, hormones, cancer markers such as PSA, cardiac markers (Troponin I, Troponin T, CKMB and α-fetoprotein) is particularly suited to the present invention. In addition, the sample may be whole blood. Thus, although the devices and methods of the present invention are suitable for assaying various body fluids, including urine, saliva, sweat or mucus for presence of particular analytes, it is particularly suited for assays in which red blood cells are present in the testing fluid and where only a small sample volume, such as a finger prick, is available for testing.

### EXAMPLES

The examples, which are intended to be purely exemplary of the invention and should, therefore, not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, sizes, etc.) but some experimental errors and deviations should be accounted for.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit or scope of the present invention. All such modifications are intended to be within the scope of the claims of the present invention.

To discover the best materials and conditions for conducting lateral flow assays for determination or quantitation of analytes in samples containing cells, such as whole blood samples containing red blood cells (“RBCs”), including determining the membrane or combination of membranes for filtering filter cells and fluid, such as plasma, in a sample, the following experiments were conducted using the instrument (“ReLIA”) and cassettes as described in U.S. Pat. No. 6,136,610 and U.S. Pat. No. 6,528,323, and as modified herein.

### Example 1

### Filtering Capability of Membranes in Absence of Agglutinins

The test strip as shown in FIG. 2 was constructed with different membranes as the sample filter. The filtering membranes tested were all obtained from Ahlstrom Filtration, Inc. (USA) and include: cellulose absorbent grade 111, glass fibers grade #141 and grade #142, Cytosep grades 1660, 1661, 1662 and 1663. A sample containing whole blood was applied in Port-1 as shown in FIG. 1. The migration speed of plasma on the nitrocellulose chromatographic strip was observed. Results were obtained as set forth in Table 2.

TABLE 2
Blood Filtering Membrane Without Anti-hRBC

| | | | | | | Time of RBC |
|---|---|---|---|---|---|---|
| | | Volume | | Migrate | | appearance |
| Membrane | Sample | μl | RBC Leaking | mm/min | Background | on NC |
| Grade 111 | Whole Blood | 100 | Yes | ≦16 | Red | 8 min |
| Cellulose | | 100 | Yes | ≦16 | Red | 8 min |
| | | 100 | Yes | ≦16 | Red | 8 min |
| | | 100 | Yes | ≦16 | Red | 8 min |
| | | 100 | Yes | ≦16 | Red | 8 min |
| Grade 141 | Whole Blood | 100 | Yes | ≦16 | Red | 3 min |
| Glass Fiber | | 100 | Yes | ≦16 | Red | 3 min |
| | | 100 | Yes | ≦16 | Red | 3 min |
| | | 100 | Yes | ≦16 | Red | 3 min |
| | | 100 | Yes | ≦16 | Red | 3 min |
| Grade 142 | Whole Blood | 100 | Yes | ≦16 | Red | 2 min |
| Glass Fiber | | 100 | Yes | ≦16 | Red | 2 min |
| | | 100 | Yes | ≦16 | Red | 2 min |
| | | 100 | Yes | ≦16 | Red | 2 min |
| | | 100 | Yes | ≦16 | Red | 2 min |
| Grade 1660 | Whole Blood | 100 | Yes | ≦16 | Red | 2 min |
| Cytosep | | 100 | Yes | ≦16 | Red | 2 min |
| | | 100 | Yes | ≦16 | Red | 2 min |
| | | 100 | Yes | ≦16 | Red | 2 min |
| | | 100 | Yes | ≦16 | Red | 2 min |
| Grade 1661 | Whole Blood | 100 | Yes | ≦16 | Red | 3 min |
| Cytosep | | 100 | Yes | ≦16 | Red | 3 min |
| | | 100 | Yes | ≦16 | Red | 3 min |
| | | 100 | Yes | ≦16 | Red | 3 min |
| | | 100 | Yes | ≦16 | Red | 3 min |
| Grade 1662 | Whole Blood | 100 | Yes | ≦16 | Red | 4 min |
| Cytosep | | 100 | Yes | ≦16 | Red | 4 min |
| | | 100 | Yes | ≦16 | Red | 4 min |
| | | 100 | Yes | ≦16 | Red | 4 min |
| | | 100 | Yes | ≦16 | Red | 4 min |
| Grade 1663 | Whole Blood | 100 | Yes | <16 | Red | 10 min |
| Cytosep | | | | | | (sticky) |
| | | 100 | Yes | <16 | Red | 10 min |
| | | | | | | (sticky) |
| | | 100 | Yes | <16 | Red | 10 min |
| | | | | | | (sticky) |
| | | 100 | Yes | <16 | Red | 10 min |
| | | | | | | (sticky) |
| | | 100 | Yes | <16 | Red | 10 min |
| | | | | | | (sticky) |

### Example 2

### Blood Filtering Capability of Membranes Pre-Treated with Anti-RBC

A ten percent (10%) TWEEN 20 solution was prepared by adding 1 g of TWEEN 20 to 9 ml of deionized water, mixing the solution, and storing the solution for about a week at room temperature.

A rabbit anti-human red blood cells antibody solution was prepared by adding 9.0825 g of Trizma Base (final concentration of 6.055 g/L), 1.7625 ml of HCl (final concentration of 1.7625 ml/L) and 1.8 g of EDTA.Na₂ (final concentration of 1.2 g/L) to 1.35 liters of deionized water. The mixture was stirred slowly until the chemical reagents were dissolved completely, about an hour. The solution was kept at room temperature for 4 hours or overnight at 4° C. The pH of the solution was adjusted to pH 8.5±0.1 by adding HCl. Rabbit anti-human red blood cell antibody (anti-hRBC) was added to the solution to a final concentration of about 0.25 mg/ml. About 0.3 ml of 10% Tween-20 solution was added to the anti-hRBC to a final concentration of 0.002%. The final solution was stored at 4° C. for 24 hr. Different membranes to be tested for use as sample filter was treated with the rabbit anti-hRBC and tested for their ability to filter fresh human whole blood samples applied to Port-1 in the configuration as exemplified in FIG. 2. Results are recorded in Table 3.

TABLE 3
Blood Filtering Membrane Pretreated With Anti-hRBC

| | | | | | | | Time of |
|---|---|---|---|---|---|---|---|
| | | | | | | | RBC |
| | Anti-RBC | | Volume | | Migrate | | shown |
| Membrane | mg/ml | Specimen | μl | RBC leaking | mm/min | Background | on NC |
| Grade 111 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 141 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 142 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 1660 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 1661 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 1662 | 0.25 | Whole Blood | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| | | | 100 | No | ≦16 | Clean | After 1 h |
| Grade 1663 | 0.25 | Whole Blood | 100 | — | — | no filtering | — |
| | | | 100 | — | — | no filtering | — |
| | | | 100 | — | — | no filtering | — |
| | | | 100 | — | — | no filtering | — |
| | | | 100 | — | — | no filtering | — |

| | | | | Testing | | | | RI = | | Time for |
|---|---|---|---|---|---|---|---|---|---|---|
| | Anti- | | RBC | Time | HC | LC | TEST | TEST | | RBC |
| Membrane | RBC | Sample | Leaking | min | Dr | Dr | Dr | Dr/LC Dr | S/CO | Leaking |
| #142 | No | HIV (+) | Yes | 30 | — | — | — | | — | 2 min |
| | No | Blood | Yes | 30 | — | — | — | | — | 2 min |
| | No | 50 μl | Yes | 30 | — | — | — | | — | 2 min |
| | No | | Yes | 30 | — | — | — | | — | 2 min |
| | No | | Yes | 30 | — | — | — | | — | 2 min |
| Buffer | Yes | | Some | 30 | — | — | — | | — | 3 min |
| Pad | Yes | | Some | 30 | — | — | — | | — | 3 min |
| | Yes | | Some | 30 | — | — | — | | — | 3 min |
| | Yes | | Some | 30 | — | — | — | | — | 3 min |
| | Yes | | Some | 30 | — | — | — | | — | 3 min |
| #142 | Yes | | No | 30 | 0.2713 | 0.2116 | 0.182 | 0.8601 | 8.603 | After 1 h |
| | Yes | | No | 30 | 0.1422 | 0.1715 | 0.1761 | 1.0268 | 10.2693 | After 1 h |
| | Yes | | No | 30 | 0.1981 | 0.1921 | 0.2282 | 1.1879 | 11.8776 | After 1 h |
| | Yes | | No | 30 | 0.2037 | 0.1843 | 0.1899 | 1.0304 | 10.3033 | After 1 h |
| | Yes | | No | 30 | 0.2287 | 0.1899 | 0.1846 | 0.9721 | 9.723 | After 1 h |

The results show that different membranes could be used for filtering red blood cells when used in conjunction with an agglutinin, such as anti-RBC antibody, and that a sample size of 50 μl is sufficient for testing.

### Example 3

### Comparison Between Using Whole Blood Versus Using Plasma

The test strip as in Example 2 was prepared and whole blood or plasma was added to the sample filter in Port-1 and the results were compared, as shown in Table 4.

TABLE 4
Comparison of Testing Results Between Whole Blood and Plasma

| | | | | | | | | | RI = | | |
|---|---|---|---|---|---|---|---|---|---|---|---|
| | Anti- | | | | | | | | TEST | | |
| | hRBC | | | RBC | Time | HC | LC | TEST | Dr/LC | | |
| Membrane | Mg/ml | Sample | Volume | Leaking | min | Dr | Dr | Dr | Dr | S/CO | Result |
| #142 | 0.25 | HIV (−) | 50 μl | No | 30 min | 0.1777 | 0.0862 | 0 | 0 | 0 | (−) |
| | 0.25 | Blood | 50 μl | No | 30 min | 0.2268 | 0.0967 | 0 | 0 | 0 | (−) |
| | 0.25 | | 50 μl | No | 30 min | 0.073 | 0.1083 | 0 | 0 | 0 | (−) |
| | 0.25 | | 50 μl | No | 30 min | 0.183 | 0.0728 | 0 | 0 | 0 | (−) |
| | 0.25 | | 50 μl | No | 30 min | 0.3277 | 0.109 | 0 | 0 | 0 | (−) |
| #142 | 0.25 | HIV (−) | 50 μl | No | 15 min | 0.4033 | 0.1926 | 0 | 0 | 0 | (−) |
| | 0.25 | Plasma | 50 μl | No | 15 min | 0.3587 | 0.1945 | 0 | 0 | 0 | (−) |
| | 0.25 | | 50 μl | No | 15 min | 0.36 | 0.1919 | 0 | 0 | 0 | (−) |
| | 0.25 | | 50 μl | No | 15 min | 0.3822 | 0.1955 | 0 | 0 | 0 | (−) |
| | 0.25 | | 50 μl | No | 15 min | 0.3759 | 0.1964 | 0 | 0 | 0 | (−) |
| #142 | 0.25 | HIV (+) | 50 μl | No | 30 min | 0.1213 | 0.1781 | 0.2014 | 1.1308 | 11.311 | (+) |
| | 0.25 | Blood | 50 μl | No | 30 min | 0.1419 | 0.1441 | 0.2181 | 1.5135 | 15.1325 | (+) |
| | 0.25 | | 50 μl | No | 30 min | 0.1781 | 0.1408 | 0.2153 | 1.5291 | 15.2911 | (+) |
| | 0.25 | | 50 μl | No | 30 min | 0.074 | 0.075 | 0.1032 | 1.3760 | 13.7626 | (+) |
| | 0.25 | | 50 μl | No | 30 min | 0.1653 | 0.1605 | 0.2142 | 1.3346 | 13.3458 | (+) |
| #142 | 0.25 | HIV (+) | 50 μl | No | 15 min | 0.2755 | 0.1423 | 0.2011 | 1.4132 | 14.13 | (+) |
| | 0.25 | Plasma | 50 μl | No | 15 min | 0.3628 | 0.18 | 0.2344 | 1.3022 | 13.0244 | (+) |
| | 0.25 | | 50 μl | No | 15 min | 0.3084 | 0.1451 | 0.2005 | 1.3818 | 13.8166 | (+) |
| | 0.25 | | 50 μl | No | 15 min | 0.3149 | 0.1468 | 0.1862 | 1.2684 | 12.6852 | (+) |
| | 0.25 | | 50 μl | No | 15 min | 0.3425 | 0.1647 | 0.2164 | 1.3139 | 13.139 | (+) |

The results show that either plasma or whole blood could be used with the test strip of the present invention, both giving substantially consistent quantifiable results.

### Example 4

### Comparison of Different Membranes as Sample Filters for Sample Application at Port-2

In this Example, the test strip as shown in FIG. 2 was constructed, but the buffer pad of FIG. 2 was substituted with one or two sample filters chosen from among the following membranes: Cytosep Grade 1661 (“1661”), Cytosep Grade 1660 (“1660”), glass fiber grade #142 (“#142”), glass fiber grade #141 (“#141”), and cellulose grade 111 (“111”) from Ahlstrom Filtration, Inc. These sample filters were tested for their ability to filter red blood cells. A conjugate pad (Millipore Corp.) and nitrocellulose membrane (Millipore Corp.) were used as shown in FIG. 2. A specified amount of blood sample was applied to Port-2. Results shown in Table 5 demonstrated that Cytosep 1661, glass fibers #141 and #142 were able to filter the sample and allow plasma to migrate to the nitrocellulose membrane, with glass fibers #141 and #142 yielding the shortest filtering time.

TABLE 5
Comparison of Different Membranes Used Singly as a Sample Filter

| | | Time for | Time for | Plasma | RBC |
|---|---|---|---|---|---|
| | Sample | Plasma to | RBC to | Migration | remained |
| Membrane | Volume | filter | leak out | Speed | on filter |
| Model | (μl) | out (sec) | (sec) | (mm/min) | after 30 min |
| 1661 | 200 | 240 | 290 | 1.8 | Yes |
| 1660 | 200 | — | — | — | |
| 141 | 200 | 90 | 280 | 16.8 | Yes |
| 142 | 200 | 98 | 395 | 16.1 | |
| 111 | 200 | — | — | — | Yes |

“—” means no plasma filtered and migrated to the Nitrocellulose membrane.

TABLE 6
Comparison of Different Membranes Used in
a Bilayer for Filtering Sample

| | | Time for | Time for | Plasma |
|---|---|---|---|---|
| | Sample | Plasma to | RBC to | Migration |
| Membrane | Volume | filter out | leak out | Speed |
| Model | (μl) | (sec) | (sec) | (mm/min) |
| 142 + 142 | 200 | 457 | 582 | 5.87 |
| 141 + 141 | 200 | 135 | 1015 | 1.95 |
| 141 + 142 | 200 | 113 | 238 | 2.93 |
| 142 + 141 | 200 | 640 | 686 | 2.26 |
| 141 + 1661 | 200 | 111 | 236 | 5.74 |
| 142 + 1660 | 200 | 247 | 409 | 3.41 |
| 142 + 1661 | 200 | 112 | 253 | 2.83 |
| 141 + 1660 | 200 | 440 | 582 | 5.71 |

Results shown in Table 6 demonstrated that all combinations of membranes tested allowed plasma to filter out of the combination sample filter at varying speed.

### Example 5

### Test of Glass Fibers Grade #141 and Grade #142 as Sample Filters for Sample Application at Port-2

In this Example, the test strip of FIG. 2 was constructed except that the buffer pad shown in FIG. 2 was substituted with a sample filter for application of sample. The sample filter used in this experiment was a single glass fiber membrane, grade #141 or grade #142, that was previously treated with 0.5 mg/ml rabbit anti-human red blood cells (An-kang Biotech, China) (“anti-hRBC”) or mouse anti-hRBC (indicated by an asterisk, *). Also, a conjugate pad (“CP”) was tested in conjunction with the glass fiber membranes. The CP was either previously treated or not treated with 0.5 mg/ml rabbit anti-human red blood cells (“anti-hRBC”) or mouse anti-hRBC. A nitrocellulose (“NC”) membrane was used as previously described. A specified amount of whole blood (200 μl) was applied to the sample filter at Port-2. Results are shown in Table 7.

The results demonstrated that all the membranes tested, whether the single CP or the combinations of #141 or #142 with CP, whether pre-treated with anti-RBC or not, were all capable of allowing plasma to filter out onto the NC membrane. The time course for the plasma to migrate to the NC ranges from about 124 seconds for the #141*+CP* combination, to 126 seconds for the #142*+CP* combination, to 130 seconds for the #141*+CP combination, to 140 for the #142*+CP combination. Plasma filtered out of the CP alone membrane in about 138 seconds. The best filtering/migrate result was with the combination of 142#+CP.”

For CP alone, RBCs leaked out of the CP relatively quickly, in about 159 seconds, and were very apparent on the NC membrane 30 min. after start of experiment. In contrast, no RBC leakage was apparent macroscopically during the course of the experiment for the #142*+CP* combination, and no RBC was macroscopically apparent on the NC membrane at the 30 min. time point after start of experiment for this combination. In comparison, the #141*+CP* combination is slightly less effective, with RBC leaking out in about 1350 seconds (22.5 min.) and a few RBCs were macroscopically apparent on the NC membrane at the 30 min. time point.

Further in comparison, the #142*+CP combination is also less effective, where CP had not been pretreated with anti-RBC, RBC started leaking out of these filters at about 1150 seconds (19.2 min) and some RBC were seen on the NC membrane at the 30 min. time point after start of experiment. For the #141*+CP combination, RBCs were observed to leak out at 680 seconds (11.3 min.) and some RBCs were observed on the NC membrane. The plasma migration speed for all membranes tested in this experiment was greater than 16 mm/min.

TABLE 7
Comparison of Combination of Membranes in Filtering RBC

| | | Time for | Time for | Plasma | RBC |
|---|---|---|---|---|---|
| | Sample | Plasma to | RBC | Migration | showed on |
| Membrane | Volume | filter | to leak out | Speed | NC after |
| Model | (μl) | out (sec) | (sec) | (mm/min) | 30 min |
| 142* + CP | 200 | 140 | 1150 | >16 | Some |
| 142* + CP* | 200 | 126 | — | >16 | No |
| 141* + CP | 200 | 130 | 680 | >16 | Some |
| 141* + CP* | 200 | 124 | 1350 | >16 | few |
| CP only | 200 | 138 | 159 | >16 | Completely |
| | | | | | leaking |

*means pretreated with 0.5 mg/ml rabbit anti-hRBC (mouse anti-hRBC)
“—” means no RBC leaking out to testing window within 30 min after blood sample applied.

The results in these Examples 1-5 show that the pore size of a single membrane alone was not determinative of whether a membrane would function well as a sample filter for the analyses herein when sample was applied at Port-2. Of the membranes tested, cellulose 111 has the smallest pore size of 1 μm (see Table 1) and was able to keep RBCs from leaking out (Table 5). However, as shown in Table 5, plasma was unable to filter out of the membrane as well. The CytoSep 1660 membrane and the glass fiber membrane #141 both have a pore size of 3 μm (see Table 1), yet plasma filtered out of glass fiber membrane #141 but not CytoSep 1660 membrane. The difference here is that the glass fiber membrane is hydrophobic and the CytoSep 1660 was less hydrophobic and more hydrophilic.

Similarly, consistent with the premise that pore size alone is not determinative of how well a membrane functions as a sample filter, the pore size of glass fiber membrane #141 was 3 μm, smaller than the pore size of glass fiber membrane #142, which was 6 μm, yet plasma filtered out of the smaller-pore-size membrane #141 more quickly, i.e., in 90 seconds compared to 98 seconds for the larger-pore-size membrane #142 (Table 5). RBCs leaked out of the #141 membrane faster than for the #142 membrane, i.e., 280 seconds versus 395 seconds. Plasma migration rate was faster for the #141 membrane (16.8 mm/min.) than the #142 membrane (16.1 mm/min.).

The use of two sample filters instead of one generally slowed down the plasma migration speed for the glass fiber membranes from about 16 mm/min (Table 5) to a range of about 1.95 to about 5.87 mm/min (Table 6). RBCs still leaked out of these bi-layered filters (Table 6). For the combination of #141+#141, it took 1015 seconds (16.9 min.) for the RBCs to leak out onto the NC membranes and 135 seconds (2.3 min.) for the plasma to filter out (Table 6). For the #142+#141 combination, where the #142 membrane is on top of the #141 membrane, RBCs leaked out in 686 seconds (11.4 min.) but took the plasma a much longer time to filter out 640 seconds (10.7 min.) (Table 6). For the combination of #141+#142, where #141 membrane is on top of #142 membrane, plasma filtered out more quickly in 113 seconds (1.9 min.), but RBC leaked out more quickly as well in 238 seconds (about 4 min.) (Table 6).

When anti-RBC antibodies were used with any of the sample filter membranes tested (Table 3), all membranes tested exhibited good RBC filtering capability in that no apparent RBC leakage took place within a 30 min. window. Background noise on the NC membrane was low. Plasma migration speed was less than 16 mm/min. For CytoSep 1663, no plasma filtering was apparent. Quantitative analysis of blood containing HIV was possible with glass fiber membrane #142 pretreated with anti-RBC (Table 3). Table 3 also shows that a sample size of 50 μl is sufficient to obtain quantitative results from the assay.

The combination of #141*+CP (Table 7), where #141 was pre-treated with anti-RBC antibody, worked better in filtering out RBC than untreated #141 alone (Table 5). Similarly #142*+CP worked better in filtering out RBC than untreated #142 alone. In this experiment, 142*+CP was most efficient in filtering RBC and plasma.

### Example 6

### Test of Efficiency of Plasma and RBC Filtering in Presence of Anti-Coagulants in the Blood Sample

Glass fiber membrane #142 was used as the sample filter in this experiment. Membrane #142 was pretreated with 0.25 mg/ml of rabbit anti-human RBC as previously described. This membrane was then pretreated with anti-coagulants (disodium EDTA: 1.5 mg/ml, trisodium citrate: 3.5 mg/ml or Heparin, sodium: 0.1 mg/ml) or not prior to use. Blood sample was also pretreated with anti-coagulants (disodium EDTA: 1.5 mg/ml, trisodium citrate: 3.5 mg/ml or Heparin, sodium: 0.1 mg/ml) or not prior to application onto the sample filter. Blood sample was applied onto sample filter in Port-2 as described above. Results are set forth in Tables 8 and 9.

TABLE 8
Efficiency of Plasma Filtering In Absence of Anticoagulant on #142

| | Whole Blood Volume | | | | | | | | |
|---|---|---|---|---|---|---|---|---|---|
| | 50 μl | | | 75 μl | | | 150 μl | | |
| #142 | | Plasma | | | Plasma | | | Plasma | |
| without | RBC | Migrate | RBC | RBC | Migrate | RBC | RBC | Migrate | RBC |
| Anticoagulant | leaking | mm/min | remained | leaking | mm/min | remained | leaking | mm/min | remained |
| Blood without | No | ≦16 | Few | No | >16 | Some | No | >16 | More |
| anticoagulant | | | | | | | | | |
| Blood with | No | ≦16 | Few | No | >16 | Some | No | >16 | More |
| EDTANa₂ | | | | | | | | | |
| added | | | | | | | | | |
| Blood with | No | ≦16 | Few | No | >16 | Some | No | >16 | More |
| Citra-Na added | | | | | | | | | |
| Blood with | No | ≦16 | Few | No | >16 | Some | No | >16 | More |
| Heparin | | | | | | | | | |
| Sodium added | | | | | | | | | |

TABLE 9
Efficiency Plasma Filtering In Presence of Anticoagulant on #142

| | Whole Blood Volume | | | | | | | | |
|---|---|---|---|---|---|---|---|---|---|
| #142 Pretreated | 50 μl | | | 75 μl | | | 150 μl | | |
| with | | Plasma | | | Plasma | | | Plasma | |
| anticoagulant | RBC | Migrate | RBC | RBC | Migrate | RBC | RBC | Migrate | RBC |
| reagents | leaking | mm/min | remained | leaking | mm/min | remained | leaking | mm/min | remained |
| Blood without | No | ≦16 | Few | No | >16 | Some | No | >16 | More |
| anticoagulant | | | | | | | | | |
| Blood with | No | ≦16 | Few | No | >16 | Some | No | >16 | More |
| EDTANa₂ | | | | | | | | | |
| added | | | | | | | | | |
| Blood with Citra- | No | ≦16 | Few | No | >16 | Some | No | >16 | More |
| Na added | | | | | | | | | |
| Blood with | No | ≦16 | Few | No | >16 | Some | No | >16 | More |
| Heparin Sodium | | | | | | | | | |
| added | | | | | | | | | |

The results show that anti-coagulants did not affect the plasma filtering efficiency when applied either on the sample filter or in the blood sample in a bilateral flow assay.

### Example 7

### Effect of RBC Volume in Quantitative Assay

To determine whether RBC volume affects the accuracy of qualitative testing in lateral stop-flow assays, the following experiments were conducted. Whole blood that was positive for hepatitis B surface antigen (“HBsAg”) with a tested hematocrit of 44% was aliquoted to 1 ml/tube. The RBC volume in these aliquots was taken as 100%. The RBC volume was then increased or decreased up to 40% through removal or addition of plasma from the same blood sample after spinning the blood sample at 800×g for 15 min. Glass fiber membrane #142 was pretreated with 0.5 mg/ml of mouse anti-human RBC and used as the sample filter at Port-2 in a HBsAg test cassette. The lateral flow assay was conducted by application of sample to the sample filter in Port-2. The lateral flow assay was conducted as before. In this configuration, a fluid collector was placed under the sample filter in a test strip as shown in FIG. 3, where the fluid collector comprises a glass fiber membrane like the conjugate pad, but without the colloidal gold labeled antigen or antibody of the conjugate pad. Results are shown in Table 10.

TABLE 10
Effect of RBC Volume in Quantitative HBsAg Assay

| | | Migration | HBsAg | | | | |
|---|---|---|---|---|---|---|---|
| | Sample | Speed | Concentration | Average 1 | Average 2 | Standard | |
| | Volume (μl) | (mm/min) | (ng/ml) | (ng/ml) | (ng/ml) | Deviation | CV |
| Normal = | 200 | >16 | 18.7 | 18.475 | 17.36 | 0.72 | 4.1% |
| 100% | | | | | | | |
| Normal = | 200 | >16 | 19.4 | | | | |
| 100% | | | | | | | |
| Normal = | 200 | >16 | 18.6 | | | | |
| 100% | | | | | | | |
| Normal = | 200 | >16 | 17.2 | | | | |
| 100% | | | | | | | |
| Testing CV | | | 4.99% | | | | |
| 10% Higher | 200 | >16 | 16.7 | 16.925 | | | |
| 10% Higher | 200 | >16 | 16.5 | | | | |
| 10% Higher | 200 | >16 | 19.3 | | | | |
| 10% Higher | 200 | >16 | 15.2 | | | | |
| Testing CV | | | 10.1% | | | | |
| 20% Higher | 200 | ≧16 | 18.2 | 16.9 | | | |
| 20% Higher | 200 | ≧16 | 16.7 | | | | |
| 20% Higher | 200 | ≧16 | 15.4 | | | | |
| 20% Higher | 200 | ≧16 | 17.3 | | | | |
| Testing CV | | | 7.0% | | | | |
| 30% Higher | 200 | <16 | 15.8 | 17.3 | | | |
| 30% Higher | 200 | <16 | 15.6 | | | | |
| 30% Higher | 200 | <16 | 20.8 | | | | |
| 30% Higher | 200 | <16 | 17.1 | | | | |
| Testing CV | | | 13.9% | | | | |
| 10% Lower | 200 | >16 | 16.8 | 16.15 | | | |
| 10% Lower | 200 | >16 | 16 | | | | |
| 10% Lower | 200 | >16 | 16.2 | | | | |
| 10% Lower | 200 | >16 | 15.6 | | | | |
| Testing CV | | | 3.1% | | | | |
| 20% Lower | 200 | >16 | 17.3 | 16.98 | | | |
| 20% Lower | 200 | >16 | 18.6 | | | | |
| 20% Lower | 200 | >16 | 16.1 | | | | |
| 20% Lower | 200 | >16 | 15.9 | | | | |
| Testing CV | | | 7.3% | | | | |
| 30% Lower | 200 | >16 | 16.7 | 17.9 | | | |
| 30% Lower | 200 | >16 | 19 | | | | |
| 30% Lower | 200 | >16 | 18.2 | | | | |
| 30% Lower | 200 | >16 | 17.7 | | | | |
| Testing CV | | | 5.4% | | | | |
| 40% Lower | 200 | >16 | 18.5 | 17.48 | | | |
| 40% Lower | 200 | >16 | 17.6 | | | | |
| 40% Lower | 200 | >16 | 16.4 | | | | |
| 40% Lower | 200 | >16 | 17.4 | | | | |
| Testing CV | | | 5.0% | | | | |
| Plasma | 200 | >16 | 19.3 | 18.1 | | | |
| Plasma | 200 | >16 | 17.6 | | | | |
| Plasma | 200 | >16 | 18.7 | | | | |
| Plasma | 200 | >16 | 16.8 | | | | |
| Testing CV | | | 6.2% | | | | |

The HBsAg concentration for a specified plasma volume was determined in 4 independent assays and averaged to produce Average 1. The average HBsAg concentration for each of the various plasma volumes tested were then averaged to produce Average 2. The observed coefficient of variation (“CV”) was less than 5% demonstrating that there was little difference between the results at the different red cell volumes.

Similarly, whole blood sample from a thyroid stimulating hormone (“TSH”) abnormal patient, having a TSH concentration of 28 μIU/ml as tested by radioimmunoassay, and a hematocrit of 44% was aliquoted to 1 ml/tube. This RBC volume was taken as 100%. The RBC volume was increased or decreased up to 40% through removing or adding plasma from the same blood sample after spinning the blood sample at 800×g for 15 min. Glass fiber membrane #142 was pretreated with 0.5 mg/ml of mouse anti-human RBC and was used as the sample filter at both Port-1 and Port-2 in a TSH test cassette. Blood sample was applied first to Port-1 and then to Port-2 in the volumes specified in Table 11. In this example, one sample was run in four different cassettes and a fluid collector was placed under the sample filter in a test strip as shown in FIG. 3, where the fluid collector comprises a glass fiber membrane like the conjugate pad, but without the colloidal gold labeled antigen or antibody of the conjugate pad.

TABLE 11
Effect of RBC Volume in Quantitative TSH Assay

| | | | | | | | RI = | | | | | |
|---|---|---|---|---|---|---|---|---|---|---|---|---|
| | | Vol. in | Test | | | | TEST | | | | | |
| | Vol. | Port-2 | Time | HC | LC | TEST | Dr/LC | Conc. | Average 1 | Average 2 | Standard | |
| Sample | In Port-1 (μl) | (μl) | (min) | Dr | Dr | Dr | Dr | (μIU/ml) | (μIU/ml) | (μIU/ml) | Deviation | CV |
| Normal | 50 | 150 | 30 | 0.3679 | 0.4733 | 0.3456 | 0.7302 | 29.66 | 28.09 | 28.5758 | 2.1258 | 7.44% |
| | | | | 0.4209 | 0.4736 | 0.3368 | 0.7111 | 28.29 | | | | |
| | | | | 0.4367 | 0.5451 | 0.4117 | 0.7553 | 31.56 | | | | |
| | | | | 0.4582 | 0.5414 | 0.3797 | 0.7013 | 27.6 | | | | |
| | | | | 0.3017 | 0.4605 | 0.2929 | 0.6360 | 23.34 | | | | |
| 10% | 50 | 150 | 30 | 0.316 | 0.5106 | 0.3958 | 0.7752 | 33.13 | 28.858 | | | |
| Higher | | | | 0.3918 | 0.6377 | 0.4369 | 0.6851 | 26.49 | | | | |
| | | | | 0.3019 | 0.4654 | 0.3739 | 0.8034 | 35.49 | | | | |
| | | | | 0.3851 | 0.6016 | 0.3954 | 0.6572 | 24.66 | | | | |
| | | | | 0.3654 | 0.5234 | 0.3428 | 0.6549 | 24.52 | | | | |
| 20% | 50 | 150 | 30 | 0.2492 | 0.4968 | 0.3521 | 0.7087 | 28.12 | 33.096 | | | |
| Higher | | | | 0.1325 | 0.4081 | 0.3378 | 0.8277 | 37.64 | | | | |
| | | | | 0.4036 | 0.4981 | 0.3921 | 0.7872 | 34.12 | | | | |
| | | | | 0.2374 | 0.4832 | 0.3663 | 0.7581 | 31.77 | | | | |
| | | | | 0.3286 | 0.4739 | 0.3714 | 0.7837 | 33.83 | | | | |
| 30% | 50 | 150 | 30 | 0.285 | 0.5246 | 0.3487 | 0.6647 | 25.14 | 25.976 | | | |
| Higher | | | | 0.3095 | 0.5654 | 0.3776 | 0.6678 | 25.35 | | | | |
| | | | | 0.387 | 0.5638 | 0.3756 | 0.6662 | 25.24 | | | | |
| | | | | 0.3331 | 0.5218 | 0.3613 | 0.6924 | 26.99 | | | | |
| | | | | 0.4314 | 0.4937 | 0.3431 | 0.6950 | 27.16 | | | | |
| 10% | 50 | 150 | 30 | 0.4032 | 0.58 | 0.4178 | 0.7203 | 28.95 | 30.118 | | | |
| Lower | | | | 0.4378 | 0.4639 | 0.3431 | 0.7396 | 30.37 | | | | |
| | | | | 0.4119 | 0.534 | 0.406 | 0.7603 | 31.95 | | | | |
| | | | | 0.3408 | 0.5217 | 0.3823 | 0.7328 | 29.86 | | | | |
| | | | | 0.403 | 0.5726 | 0.4165 | 0.7274 | 29.46 | | | | |
| 20% | 50 | 150 | 30 | 0.2859 | 0.4134 | 0.5012 | 1.2124 | 28.8 | 27.944 | | | |
| Lower | | | | 0.3542 | 0.3538 | 0.5372 | 1.5184 | 28.03 | | | | |
| | | | | 0.1217 | 0.4228 | 0.5584 | 1.3207 | 26.65 | | | | |
| | | | | 0.3955 | 0.3661 | 0.5727 | 1.5643 | 26.31 | | | | |
| | | | | 0.1942 | 0.3896 | 0.4754 | 1.2202 | 29.93 | | | | |
| 30% | 50 | 150 | 30 | 0.4156 | 0.531 | 0.3725 | 0.7015 | 27.62 | 28.176 | | | |
| Lower | | | | 0.4138 | 0.5416 | 0.3874 | 0.7153 | 28.58 | | | | |
| | | | | 0.4028 | 0.5107 | 0.3742 | 0.7327 | 29.85 | | | | |
| | | | | 0.3895 | 0.5227 | 0.3686 | 0.7052 | 27.87 | | | | |
| | | | | 0.3955 | 0.5437 | 0.3763 | 0.6921 | 26.96 | | | | |
| 40% | 50 | 150 | 30 | 0.3898 | 0.5024 | 0.3631 | 0.7227 | 29.12 | 28.732 | | | |
| Lower | | | | 0.4023 | 0.5343 | 0.412 | 0.7711 | 32.81 | | | | |
| | | | | 0.369 | 0.5512 | 0.3706 | 0.6724 | 25.64 | | | | |
| | | | | 0.3775 | 0.4997 | 0.3581 | 0.7166 | 28.68 | | | | |
| | | | | 0.4182 | 0.5448 | 0.3806 | 0.6986 | 27.41 | | | | |
| Plasma | 50 | 150 | 30 | 0.4 | 0.4439 | 0.3034 | 0.6835 | 26.39 | 26.192 | | | |
| | | | | 0.361 | 0.4208 | 0.2795 | 0.6642 | 25.12 | | | | |
| | | | | 0.3539 | 0.4248 | 0.2846 | 0.6700 | 25.49 | | | | |
| | | | | 0.4089 | 0.4244 | 0.3018 | 0.7111 | 28.29 | | | | |
| | | | | 0.3799 | 0.448 | 0.3014 | 0.6728 | 25.67 | | | | |

The TSH concentration for a specified plasma volume was determined in 5 independent assays and averaged to produce Average 1. The average TSH concentration for each of the various plasma volumes tested was then averaged to produce Average 2. The observed coefficient of variation (“CV”) was less than 8% demonstrating that there was little difference between the TSH results at the different red cell volumes.

The above results show that the present assay, using samples containing fluid and cells, is volume independent.

### Example 8

### Effect of Red Blood Cell Volume in Qualitative Assay

To determine whether presence of RBC affects the accuracy of qualitative or quantitative testing in lateral bidirectional flow assay, the following experiment was conducted. HIV-positive whole blood with a tested RBC hematocrit of 45.3% was aliquoted to 1 ml/tube. This RBC volume was taken as 100%. The RBC volume was increased or decreased by removing or adding plasma from the same blood sample after spinning the blood sample at 800×g for 15 min. Glass fiber membrane #142 was pretreated with 0.25 mg/ml of rabbit anti-hRBC and used as a sample filter at Port-1 in a HIV test cassette. Results are recorded in Table 12.

TABLE 12
Effect of Red Blood Cells Volume in HIV Assay

| | | | | | RI = | | | | | |
|---|---|---|---|---|---|---|---|---|---|---|
| | Test | HC | LC | TEST | TEST | | | | | |
| RBC Vol. | Time | Dr | Dr | Dr | Dr/LC Dr | S/CO | AVE | AVE 2 | SD | CV |
| Normal = | 30 min | 0.2341 | 0.1272 | 0.2059 | 1.6187 | 16.18 | 19.69 | 20.0019 | 1.8855 | 9.43% |
| 100% | | | | | | | | | | |
| Normal = | 30 min | 0.2066 | 0.0941 | 0.2123 | 2.2561 | 22.57 | | | | |
| 100% | | | | | | | | | | |
| Normal = | 30 min | 0.2824 | 0.0942 | 0.1914 | 2.0318 | 20.32 | | | | |
| 100% | | | | | | | | | | |
| 10% higher | 30 min | 0.3702 | 0.1324 | 0.2564 | 1.9366 | 19.36 | 19.61 | | | |
| 10% higher | 30 min | 0.225 | 0.1193 | 0.2241 | 1.8785 | 18.78 | | | | |
| 10% higher | 30 min | 0.0961 | 0.1044 | 0.216 | 2.0690 | 20.69 | | | | |
| 20% higher | 30 min | 0.0503 | 0.1144 | 0.1752 | 1.5315 | 15.32 | 17.9233 | | | |
| 20% higher | 30 min | 0.0753 | 0.136 | 0.2474 | 1.8191 | 18.19 | | | | |
| 20% higher | 30 min | 0.0747 | 0.1107 | 0.2243 | 2.0262 | 20.26 | | | | |
| 30% higher | 30 min | 0.1036 | 0.1056 | 0.2463 | 2.3324 | 23.32 | 17.3633 | | | |
| 30% higher | 30 min | 0.2257 | 0.1545 | 0.2548 | 1.6492 | 16.49 | | | | |
| 30% higher | 30 min | 0.2486 | 0.1975 | 0.2425 | 1.2278 | 12.28 | | | | |
| 10% lower | 30 min | 0.2859 | 0.0954 | 0.2555 | 2.6782 | 26.78 | 22.8267 | | | |
| 10% lower | 30 min | 0.1217 | 0.12 | 0.3136 | 2.6133 | 26.13 | | | | |
| 10% lower | 30 min | 0.3955 | 0.1297 | 0.202 | 1.5574 | 15.57 | | | | |
| 20% lower | 30 min | 0.3806 | 0.1136 | 0.254 | 2.2359 | 22.36 | 18.7033 | | | |
| 20% lower | 30 min | 0.2666 | 0.1149 | 0.1925 | 1.6754 | 16.75 | | | | |
| 20% lower | 30 min | 0.3167 | 0.1075 | 0.1828 | 1.7005 | 17 | | | | |
| 30% lower | 30 min | 0.3931 | 0.1031 | 0.2531 | 2.4549 | 24.55 | 20.2467 | | | |
| 30% lower | 30 min | 0.4278 | 0.1615 | 0.2365 | 1.4644 | 14.64 | | | | |
| 30% lower | 30 min | 0.1701 | 0.1014 | 0.2185 | 2.1548 | 21.55 | | | | |
| 40% lower | 30 min | 0.2957 | 0.0805 | 0.2067 | 2.5677 | 25.67 | 21.3167 | | | |
| 40% lower | 30 min | 0.3816 | 0.1069 | 0.188 | 1.7587 | 17.59 | | | | |
| 40% lower | 30 min | 0.3317 | 0.0905 | 0.1873 | 2.0696 | 20.69 | | | | |
| Plasma | 15 min | 0.3661 | 0.0844 | 0.1688 | 2.0000 | 20 | 22.3367 | | | |
| Plasma | 15 min | 0.2843 | 0.0741 | 0.1689 | 2.2794 | 22.79 | | | | |
| Plasma | 15 min | 0.3869 | 0.0898 | 0.2175 | 2.4220 | 24.22 | | | | |

The results reported for HC, LC and TC are density of reflectance (DR). The results show that there was no obvious effect of the presence of RBC in S/CO (S means signal; CO means cutoff). In any non-quantitative, i.e., qualitative assay, S/CO>1 means positive because CO is determined by the average of a large quantity of negative samples. It represents the background (in an HIV test) on HIV testing when a whole blood sample was applied as compared to application of a plasma sample when sample was applied on #142 membrane pretreated with anti-hRBC at Port-1 in a Bi-directional flow assay. The test band is measured as RI (ration of the relative density of reflection) which is the basic measurement used. To avoid the variables between cassettes of the same lot, the Dr (reflection density) of one of the control bands (HC or LC) is used to calculate the RI of test band in the formula: RI (Test band)=Test Dr/HC Dr (or LC Dr). This provides higher reproducibility as the effect of variables on both control bands and the test band are balanced by RI.

### Example 9

### Effect of Gold Conjugate Coating Methods on Accuracy of Assay

To determine the effect of different coating methods for coating conjugates on the conjugate pad, the following experiment was conducted using conjugate pads coated with gold-labeled anti-human IgG or gold labeled anti-HBsAg using a rinse coating procedure or a Bio-jet coating method. Glass fiber membrane #142 pretreated with 0.25 mg/ml of rabbit anti-hRBC was used as the sample fitter. Gold-labeled anti-human IgG or anti-HBsAg coated on a glass conjugate pad (Pall Specialty Materials) by Bio-jet or rinse methods were obtained from Pall Specialty. The experiment was conducted using HIV positive whole blood in a HIV test cassette and HBsAg positive whole blood in an HBsAg test cassette. Blood sample was applied on the sample filter at Port-2 for the HBsAg assay (as described in Example 7 above and on the sample filter at port 1 for the HIV assay (as described in Example 3 above) and the assays were conducted as before. Results are shown in Tables 13 and 14.

TABLE 13
HIV Test (Conjugate coated by Bio-jet or Rinse)

| | | | | | | | RI = | | |
|---|---|---|---|---|---|---|---|---|---|
| | | | | HC | LC | TEST | TEST Dr/LC | | AV |
| Coated | Sample | Volume | Background | Dr | Dr | Dr | Dr | S/CO | S/CO |
| Resin | HIV (+) | 50 μl | Clean | 0.1564 | 0.1785 | 0.1736 | 0.9725 | 9.7255 | Average |
| Resin | Blood | 50 μl | Clean | 0.1458 | 0.1793 | 0.1806 | 1.0073 | 10.0725 | 9.614 |
| Resin | | 50 μl | Clean | 0.1135 | 0.2106 | 0.2094 | 0.9943 | 9.943 | Standard |
| Resin | | 50 μl | Clean | 0.1356 | 0.1883 | 0.1683 | 0.8938 | 8.9379 | Deviation |
| Resin | | 50 μl | Clean | 0.1079 | 0.1872 | 0.1758 | 0.9391 | 9.391 | 0.457 |
| | | | | | | | | | CV |
| | | | | | | | | | 4.76% |
| Jet | | 50 μl | Clean | 0.1456 | 0.2281 | 0.2107 | 0.9237 | 9.2372 | Average |
| Jet | | 50 μl | Clean | 0.1175 | 0.1758 | 0.1658 | 0.9431 | 9.4312 | 9.456 |
| Jet | | 50 μl | Clean | 0.1334 | 0.1606 | 0.1708 | 1.0635 | 10.6351 | Standard |
| Jet | | 50 μl | Clean | 0.1238 | 0.2301 | 0.2152 | 0.9352 | 9.3525 | Deviation |
| Jet | | 50 μl | Clean | 0.1034 | 0.1898 | 0.1637 | 0.8625 | 8.6249 | 0.731 |
| | | | | | | | | | CV |
| | | | | | | | | | 7.7% |

TABLE 14
HBsAg Test (Conjugate coated by Bio-jet or Rinse)

| | | | | RI = | | | | | | RI = | |
|---|---|---|---|---|---|---|---|---|---|---|---|
| | HC | LC | HBsAg | HBsAg Dr/ | | | HC | LC | HBsAg | HBsAg Dr/ | |
| Jet | Dr | Dr | Dr | HC Dr | ng/ml | Resin | Dr | Dr | Dr | HC Dr | ng/ml |
| 1 | 0.2153 | 0.1433 | 0.0153 | 0.0711 | 3.9 | 1 | 0.23 | 0.25 | 0.011 | 0.0478 | 2.6 |
| 2 | 0.1879 | 0.1163 | 0.0144 | 0.0766 | 4.3 | 2 | 0.31 | 0.33 | 0.019 | 0.0613 | 3.4 |
| 3 | 0.1759 | 0.1245 | 0.0167 | 0.0949 | 5.5 | 3 | 0.34 | 0.34 | 0.017 | 0.0500 | 2.5 |
| 4 | 0.1419 | 0.0845 | 0.0135 | 0.0951 | 5.5 | 4 | 0.26 | 0.29 | 0.013 | 0.0500 | 2.6 |
| 5 | 0.1985 | 0.1242 | 0.0176 | 0.0887 | 5.1 | 5 | 0.36 | 0.35 | 0.02 | 0.0556 | 3 |
| 6 | 0.202 | 0.1267 | 0.014 | 0.0693 | 3.8 | 6 | 0.35 | 0.34 | 0.017 | 0.0486 | 2.6 |
| 7 | 0.1898 | 0.1291 | 0.0152 | 0.0801 | 4.5 | 7 | 0.34 | 0.33 | 0.019 | 0.0559 | 3 |
| 8 | 0.1764 | 0.1456 | 0.0143 | 0.0811 | 4.6 | 8 | 0.43 | 0.36 | 0.021 | 0.0488 | 2.5 |
| 9 | 0.2306 | 0.1917 | 0.0206 | 0.0893 | 5.1 | 9 | 0.36 | 0.36 | 0.018 | 0.0500 | 2.6 |
| 10 | 0.192 | 0.1168 | 0.0139 | 0.0724 | 4 | 10 | 0.35 | 0.34 | 0.019 | 0.0543 | 2.9 |
| 11 | 0.1895 | 0.1219 | 0.017 | 0.0897 | 5.2 | 11 | 0.27 | 0.28 | 0.016 | 0.0593 | 3.1 |
| 12 | 0.2355 | 0.1692 | 0.021 | 0.0892 | 5.1 | 12 | 0.39 | 0.36 | 0.02 | 0.0513 | 2.7 |
| 13 | 0.2369 | 0.1589 | 0.0156 | 0.0659 | 3.6 | 13 | 0.38 | 0.35 | 0.019 | 0.0500 | 2.7 |
| 14 | 0.2041 | 0.1543 | 0.015 | 0.0735 | 4.1 | 14 | 0.32 | 0.29 | 0.013 | 0.0406 | 2.1 |
| 15 | 0.2294 | 0.1633 | 0.0175 | 0.0763 | 4.3 | 15 | 0.37 | 0.33 | 0.019 | 0.0514 | 2.7 |
| 16 | 0.1611 | 0.0947 | 0.0146 | 0.0906 | 5.2 | 16 | 0.32 | 0.28 | 0.014 | 0.0438 | 2.3 |
| 17 | 0.2204 | 0.1518 | 0.0218 | 0.0989 | 5.8 | 17 | 0.2 | 0.22 | 0.013 | 0.0650 | 3.6 |
| 18 | 0.1785 | 0.1272 | 0.0151 | 0.0846 | 4.8 | 18 | 0.33 | 0.27 | 0.017 | 0.0515 | 2.7 |
| 19 | 0.2607 | 0.1911 | 0.0231 | 0.0886 | 5.1 | 19 | 0.32 | 0.26 | 0.019 | 0.0594 | 3.1 |
| 20 | 0.1884 | 0.1161 | 0.0162 | 0.0860 | 4.9 | 20 | 0.28 | 0.3 | 0.015 | 0.0536 | 2.9 |
| AV | 0.2007 | 0.1376 | 0.0166 | 0.0831 | 4.72 | AV | 0.3255 | 0.3115 | 0.0170 | 0.0524 | 2.780 |
| SD | 0.0289 | 0.0285 | 0.0028 | 0.0095 | 0.63 | SD | 0.0556 | 0.0417 | 0.0029 | 0.0058 | 0.354 |
| CV | | | | 11.5% | 13.0% | CV | | | | 11.2% | 12.8% |

DR stands for density of reflectance which is calculated the same way as optical density (OD), except DR is for reflected light. DR is the raw data generated by the ReLIA machine. The CV on the ng/ml result is the same for rinse and Biojet coated conjugate, so the testing accuracies are the same.

### Example 10

### Effect of Sample Volume on Assay

To determine the effect of sample volume on the accuracy of the present assay, the following experiment was conducted. Glass fiber membrane #142 pretreated with 0.25 mg/ml of rabbit anti-hRBC was used as the sample filter in Port-1. HIV-positive whole blood containing a RBC volume of 45.3% was used as sample in a HIV testing cassette. HBsAg-positive whole blood, having a hematocrit of 44%, was used as sample in an HBsAg testing cassette. Varying sample volumes were applied to the sample filter at Port-2. The assay was conducted as before. Briefly, the HIV assay with whole blood as sample was performed with the sample added to port 1. The sample filter in port 1 consists of Glass fiber membrane #142 pretreated with 0.25 mg/ml of rabbit anti-hRBC. After the sample had flowed down the strip and stopped, buffer was added to port 2. Results are recorded in Table 15.

TABLE 15
Comparison of HIV Testing with Varying Volume of Blood Specimen

| | | | | | | | | RI = | | | | |
|---|---|---|---|---|---|---|---|---|---|---|---|---|
| | Sample | Migration | | | | | | TEST | | | | Test |
| | Vol. | Rate | RBC | Background | HC | LC | TEST | Dr/LC | | | | Time |
| Sample | μl | mm/min | remain | on NC | Dr | Dr | Dr | Dr | S/CO | Avg | CV | (min) |
| HIV (+) | 30 | .16 | No | Clean | 0.4752 | 0.226 | 0 | 0 | 0 | 0 | — | 20 |
| Blood | 30 | .16 | No | Clean | 0.4154 | 0.2432 | 0 | 0 | 0 | | | |
| | 30 | .16 | No | Clean | 0.4163 | 0.2317 | 0 | 0 | 0 | | | |
| | 40 | .16 | No | Clean | 0.2936 | 0.1979 | 0.2043 | 1.0323 | 10.3223 | 9.0592 | 12.9% | 20 |
| | 40 | .16 | No | Clean | 0.2697 | 0.1958 | 0.1731 | 0.8841 | 8.8396 | | | |
| | 40 | .16 | No | Clean | 0.2994 | 0.1986 | 0.1592 | 0.8016 | 8.0156 | | | |
| | 40 | .16 | No | Clean | 0.371 | 0.225 | 0.2507 | 1.1142 | 9.1408 | 9.5644 | 8.86% | 30 |
| | 40 | .16 | No | Clean | 0.1701 | 0.1983 | 0.209 | 1.054 | 10.5396 | | | |
| | 40 | .16 | No | Clean | 0.3206 | 0.1955 | 0.1762 | 0.9013 | 9.0128 | | | |
| | 50 | ≦16 | (+/−) | Clean | 0.2037 | 0.1697 | 0.2164 | 1.2752 | 12.7513 | 12.0134 | 6.5% | 30 |
| | 50 | ≦16 | (+/−) | Clean | 0.2572 | 0/.1876 | 0.2271 | 1.2106 | 12.1034 | | | |
| | 50 | ≦16 | (+/−) | Clean | 0.0603 | 0.1729 | 0.1934 | 1.1186 | 11.1856 | | | |
| | 60 | .16 | (+) | Clean | 0.3271 | 0.2474 | 0.2005 | 0.8104 | 8.105 | 9.8273 | 20.42% | 30 |
| | 60 | .16 | (+) | Clean | 0.3456 | 0.1984 | 0.2387 | 1.2031 | 12.0302 | | | |
| | 60 | .16 | (+) | Clean | 0.1926 | 0.246 | 0.2299 | 0.9346 | 9.3467 | | | |
| | 70 | .16 | (+) | Clean | 0.1296 | 0.1983 | 0.1913 | 0.9647 | 9.649 | 9.6556 | 8.4% | 30 |
| | 70 | .16 | (+) | Clean | 0.1387 | 0.245 | 0.2168 | 0.8849 | 8.8477 | | | |
| | 70 | .16 | (+) | Clean | 0.1119 | 0.2401 | 0.2514 | 1.0471 | 10.4702 | | | |

In this HIV testing experiment, sample volumes of from 30 μl up to 70 μl were used. The 30 μl sample volume did not generate any readable test results, while sample volumes higher than 30 μl did. For the 40 μl samples, for example, readable results were obtained regardless of whether the test was conducted for 20 min. or 30 min. The background on the NC membrane was clean for all the sample volumes applied. Thus, a 40 μl sample volume is sufficient for running this assay, but results are not significantly different at sample sizes of 50, 60, or 70 μl.

TABLE 16
Comparison of varying specimen volume in HBsAg Testing Within
Different Assay Time. Assays were run in triplicate.

| | | | Testing results | | | | | | | | |
|---|---|---|---|---|---|---|---|---|---|---|---|
| Whole | | Plasma | 20 min | | | 30 min | | | 40 min | | |
| Blood | RBC | Migrate | RBC | HBsAg | | RBC | HBsAg | | RBC | HBsAg | |
| (μl) | Leaking | (mm/min | remained | (ng/ml) | CV % | remained | (ng/ml) | CV % | remained | (ng/ml) | CV % |
| 150 | No | >16 mm | (+/−) | 3.2 | 42% | (−) | 3.94 | 42 | (−) | 4.1 | 46% |
| 200 | No | >16 mm | (+) | 3.28 | 9% | (+/−) | 3.46 | 3 | (−) | 3.27 | 19% |
| 250 | No | >16 mm | (++) | 2.63 | 6% | (+) | 2.77 | 6 | (+/−) | 2.75 | 4% |
| 300 | No | >16 mm | (+++) | 2.77 | 6% | (++) | 2.83 | 9 | (++) | 3.13 | 10% |

| | | |
|---|---|---|
| Plasma | 30 min | CV % |
| 150 ul | 3.36 | 5% |

Results from the HBsAg testing showed that using a sample volume of 150 μl resulted in a high CV %. At sample volumes greater than 150 μl, such as 200 μl, 250 μl, or 300 μl, the CV % were in the low ranges of 6%-9% for the 20 min. assay, 3%-9% for the 30 min. assay and 4%-19% for the 40 min. assay. For the 250 μl sample volume, the CV % was in the low range of 4%-6% regardless of whether it is a 20 min., 30 min. or 40 min. assay. Thus, a 250 μl sample volume is sufficient for running this assay, but results are not significantly different at sample sizes of 200, 250, or 300 μl if the assay is run for either 20 or 30 minutes. The CV was higher (19%) for the 200 μl assay if the assay was carried out for 40 minutes rather than 20 or 30 minutes.

All alternatives described above in terms of the general format, such as the placement of absorbers, the use or omission of a sample filter or its replacement with a sample pad, the use or omission of agglutinating agents, the replacement of hydrophobic elements such as membranes with hydrophilic membranes, the placement of detectable agents in a conjugate pad or in the test strip itself, or the omission of the detectable agent from the test strip, the use or omission of buffer pads, or the placement of conjugate pads, can be applied to the specific formats, such as those of FIG. 2, 3, 4, 8, 9, 10, or 11 as long as such alternatives are consistent with the configurations of those specific formats.

### REFERENCES

Fu, G. et al. (2004). Purification and characterization of the human erythrocyte band 3 protein C-terminal domain. Biochemistry 43(6) 1633-8.
Wang, D. N. (1994). Band 3 protein: structure, flexibility and function. FEBS Lett. 346(1): 26-31.
Young, M. T. and Tanner, M. J. (2003). Distinct regions of human glycophorin A enhance human red cell anion exchanger (Band 3; AE1) transport function and surface trafficking. J. Biol. Chem. 278(35): 32954-61. Epub 2003 Jun. 17.

We claim:

1. A test strip for a lateral flow assay for detection or quantitation of at least one analyte in a sample containing a fluid comprising:
(a) a first membrane, wherein the first membrane comprises a sample filter and the sample filter comprises a first pore size and, optionally, a first agglutinating agent;
(b) optionally, a second membrane, wherein the second membrane comprises a first fluid collector and the first fluid collector comprises a second pore size, wherein the second membrane, if present, is in capillary contact with the first membrane;
(c) optionally, a third membrane, wherein the third membrane comprises a conjugate pad and the conjugate pad, if present, is in capillary contact, directly or indirectly, with the chromatographic strip, and wherein the conjugate pad comprises at least one mobilizable detectable agent, and the at least one mobilizable detectable agent is a first mobilizable detectable agent;
(d) a fourth membrane, wherein the fourth membrane comprises a chromatographic strip that comprises a first end and a second end, at least one capture band, at least one control band that optionally comprises a control agent and, optionally, at least one mobilizable detectable agent, wherein the at least one mobilizable detectable agent is a second mobilizable detectable agent, wherein the at least one capture band comprises an immobilized capture agent for capturing the at least one analyte, wherein the chromatographic strip allows lateral flow of fluid from the first end to the second end or from the second end to the first end, and wherein the chromatographic strip is in capillary contact with at least one of the first, second or third membrane, directly or indirectly;
(e) optionally, a fifth membrane comprising a buffer pad for application of sample, buffer, or reagent, wherein the fifth membrane, if present, is in capillary contact with the fourth membrane and optionally comprises a second agglutinating agent;
(f) a sixth membrane, wherein the sixth membrane comprises a first absorbent pad, and the first absorbent pad is in capillary contact with the chromatographic strip directly or indirectly;
(g) optionally, a seventh membrane, wherein the seventh membrane comprises a second absorbent pad, and the second absorbent pad, if present, is in capillary contact with the sixth membrane; and
(h) optionally, an eighth membrane, wherein the eighth membrane comprises a second fluid collector, and the second fluid collector, if present, is in capillary contact with the fourth membrane and the fifth membrane, if present; and
wherein the test strip is configured to allow detection or quantitation of the at least one analyte in the sample, and the sample contains red blood cells.

2. The test strip of claim 1, wherein the test strip comprises the second membrane and ratio of the second pore size to the first pore size is less than about 20 and is greater than about 1.

3. The test strip of claim 2, wherein the ratio of the second pore size to the first pore size is less than about 10.

4. The test strip of claim 2, wherein the ratio of the second pore size to the first pore size is about 7.

5. The test strip of claim 1, wherein the test strip comprises the second membrane and wherein at least a portion of the first membrane is situated on top of the second membrane.

6. The test strip of claim 1, wherein the test strip comprises the second membrane and the conjugate pad, and the first membrane is in capillary contact with the conjugate pad through the second membrane but does not physically touch the conjugate pad.

7. The test strip of claim 1, wherein the test strip comprises the fifth membrane, and the fifth membrane comprises a second agglutinating agent.

8. The test strip of claim 1, wherein the first absorbent pad is in capillary contact with the fifth membrane, directly or indirectly.

9. The test strip of claim 1, wherein the test strip comprises the fifth membrane, the fifth membrane comprising the second agglutinating agent, and wherein the test strip is configured for application of sample onto the fifth membrane and application of buffer onto the first membrane.

10. The test strip of claim 1, wherein the first membrane comprises the first agglutinating agent, the test strip comprises the fifth membrane, and wherein the test strip is configured for application of sample onto the first membrane and application of buffer onto the fifth membrane.

11. The test strip of claim 1, wherein the test strip comprises the fifth membrane, the first and fifth membrane comprising the first and second agglutinating agent, respectively, and wherein the test strip is configured for application of sample onto both the first and fifth membranes.

12. The test strip of claim 1, wherein the test strip is configured to operate in a manner shown in FIG. 6.

13. The test strip of claim 1, wherein the test strip is configured to operate in a manner shown in FIG. 7.

14. The test strip of claim 1, wherein the sixth or seventh membrane is situated adjacent to the sample filter and on the side opposite to the buffer pad.

15. A test strip for a lateral flow assay for detection of at least one analyte in a sample containing a fluid comprising:
(a) a first membrane comprising a sample filter, wherein the sample filter optionally comprises an agglutinating agent;
(b) a second membrane comprising a chromatographic strip, wherein the chromatographic strip includes a first end and a second end, at least one capture band that comprises an immobilized capture agent for capturing the at least one analyte, at least one control band and, optionally, a first mobilizable detectable agent, wherein the chromatographic strip supports lateral flow of fluid from the first end to the second end or from the second end to the first end, and wherein the chromatographic strip is in capillary contact with the sample filter;
(c) optionally, a third membrane comprising a conjugate pad, wherein the conjugate pad comprises a second mobilizable detectable agent, and the conjugate pad, if present, is in capillary contact with the chromatographic strip;
(d) a fourth membrane comprising a buffer pad, wherein the buffer pad is in capillary contact with the conjugate pad or the chromatographic strip;
(e) a fifth membrane comprising a first absorbent pad;
(f) optionally, a sixth membrane comprising a second absorbent pad; and
(g) optionally, a seventh membrane comprising a first fluid collector;
wherein the test strip is configured to allow detection or quantitation of the at least one analyte in the sample, and the sample contains red blood cells.

16. The test strip of claim 1 or 15, wherein the sample filter, first fluid collector, second fluid collector, conjugate pad, or fifth membrane, if present, comprises a glass fiber material.

17. The test strip of claim 1 or 15, wherein the first membrane is situated entirely on top of the second membrane.

18. The test strip of claim 1 or 15, wherein the test strip comprises a conjugate pad, and wherein the conjugate pad receives fluid from the sample filter and overlaps the chromatographic strip for a distance sufficient for fluid to pass from the conjugate pad to the chromatographic strip.

19. The test strip of claim 1 or 15, wherein the test strip comprises the first fluid collector, and the first fluid collector overlaps the chromatographic strip for a distance sufficient for fluid to pass from the sample filter to the chromatographic strip.

20. The test strip of claim 1 or 15, wherein the sample filter comprises the first agglutinating agent.

21. The test strip of claim 20, wherein the first agglutinating agent is selected from the group consisting of lectins and anti-blood-cell antibodies.

22. The test strip of claim 21, wherein the anti-blood cell antibody is an anti-Band 3 antibody or an anti-glycophorin antibody.

23. The test strip of claim 1 or 15, wherein the sample filter is capable of separating cells from fluid in the sample.

24. The test strip of claim 23, wherein the cells are red blood cells.

25. The test strip of claim 1 or 15, wherein the capture agent is an antibody or an antigen that specifically binds the analyte.

26. The test strip of claim 1 or 15, wherein the chromatographic strip comprises at least two control bands.

27. The test strip of claim 1 or 15, wherein the chromatographic strip comprises a nitrocellulose membrane.

28. The test strip of claim 1 or 15, wherein the chromatographic strip comprises particles of a polymer fused together.

29. The test strip of claim 28, wherein the polymer is polyethylene.

30. The test strip of claim 1 or 15, wherein the first or second mobilizable detectable agent comprises one selected from the group consisting of: gold, a colored agent, a fluorescent agent, a chemiluminescent agent, a bioluminescent agent, and an agent that can be combined with another agent to produce color, fluorescence, chemiluminescence, or bioluminescence.

31. The test strip of claim 30, wherein the mobilizable detectable agent comprises colloidal gold.

32. The test strip of claim 1 or 15, wherein the first or second mobilizable detectable agent comprises an antibody or an antigen.

33. The test strip of claim 1 or 15, wherein the test strip is configured in a manner shown in one of FIG. 2, 3, 4, 5, 8, or 9.

34. The test strip of claim 1 or 15, wherein the test strip comprises the third membrane.

35. The test strip of claim 1 or 15, wherein the test strip lacks a conjugate pad and the chromatographic strip comprises the second mobilizable detectable agent.

36. The test strip of claim 1 or 15, wherein the test strip is configured to support unidirectional or bidirectional fluid flow.

37. The test strip of claim 36, wherein the test strip is configured to support unidirectional flow.

38. The test strip of claim 37, wherein the test strip is configured such that the unidirectional flow is stopped flow.

39. The test strip of claim 37, wherein the test strip is configured such that the unidirectional flow is reversed flow.

40. The test strip of claim 36, wherein the test strip is configured to support bidirectional flow.

41. The test strip of claim 1 or 15 further comprising a backing that supports the test strip.

42. The test strip of claim 41, wherein the backing comprises a liquid impermeable backing.

43. The test strip of claim 1 or 15, wherein the chromatographic membrane is a high capacity protein binding membrane.

44. The test strip of claim 15, wherein the buffer pad overhangs the conjugate pad.

45. The test strip of claim 15, wherein the test strip is configured as shown in FIG. 2.

46. The test strip of claim 1 or 15, wherein the sample contains two analytes and the chromatographic strip comprises two separate capture bands, each capture band comprising an immobilized capture agent that is specific for capturing one analyte but not the other.

47. The test strip of claim 1 or 15, wherein the sample contains three analytes and the chromatographic strip comprises three separate capture bands, each capture band comprising an immobilized capture agent that is specific for capturing one analyte but not the other two.

48. The test strip of claim 1 or 15, wherein the analyte is an analyte selected from the group consisting of an antigen, an antibody, a hormone, a drug, a cell protein, a DNA, a cardiac marker, a tumor marker, a ligand, a receptor and an autoimmune disease marker.

49. The test strip of claim 48, wherein the analyte is an antigen, and the antigen is an antigen that is associated with an infectious agent.

50. The test strip of claim 49, wherein the infectious agent is selected from the group consisting of: a virus, a bacterium, a fungus, or a prion.

51. The test strip of claim 50, wherein the infectious agent is a virus, and wherein the virus is selected from the group consisting of HIV, hepatitis virus A, B, C, and D, herpes simplex virus, cytomegalovirus, papilloma virus, Ebola virus, SARS virus, Rhinovirus, and Vaccinia virus.

52. The test strip of claim 50, wherein the infectious agent is a bacterium and the bacterium is selected from the group consisting of Gram positive bacteria and Gram negative bacteria.

53. The test strip of claim 52, wherein the bacterium is selected from the group consisting of Bacillus anthracis, Escherichia coli, Helicobacter pylori, Pasteurella pestis, Salmonella species, and Shigella species.

54. The test strip of claim 48, wherein the analyte is a hormone selected from the group consisting of hCG, thyroxin, and TSH.

55. The test strip of claim 48, wherein the analyte is selected from the group consisting of a tumor marker, a cardiac marker, and an autoimmune disease marker.

56. The test strip of claim 55, wherein the analyte is a tumor marker and the tumor marker is selected from the group consisting of prostate specific antigen, carcinoembryonic antigen, and α-fetoprotein.

57. The test strip of claim 48, wherein the analyte is a cell protein.

58. The test strip of claim 48, wherein the analyte is a cardiac marker and the cardiac marker is selected from the group consisting of Troponin-I, Troponin T, Creatine kinase-MB isoforms (CK-MB), myoglobin, C-reactive protein (CRP), fatty acid binding protein (FABP), glycogen phosphorylase isoenzyme BB (GPBB), B-type natriuretic peptide (BNP) and pro-BNP.

59. The test strip of claim 1 or 15, wherein the sample filter is pretreated with a detergent prior to incorporation of the agglutinating agent.

60. A test strip for a lateral flow assay for detection of at least one analyte in a sample comprising:
(a) a chromatographic strip having a first end and a second end, the test strip including a capture band for capturing the analyte;
(b) a fluid-transmitting element in operable contact with the first end of the chromatographic strip, the fluid-transmitting element being selected from the group consisting of a sample pad and a first sample filter, the fluid-transmitted element being located so that fluid applied to the fluid-transmitting element passes through the fluid-transmitting element and is applied to the chromatographic strip;
(c) at least one absorbent pad in operable contact with the fluid-transmitting element;
(d) optionally, a conjugate pad in operable contact with the second end of the chromatographic strip, the conjugate pad including a labeled specific binding partner for the analyte;
(e) a fluid collector in operable contact with either the conjugate pad, if present, or with the second end of the chromatographic strip, if the conjugate pad is not present, so that fluid applied to the fluid collector passes through the fluid collector to the conjugate pad, if present, or to the second end of the chromatographic strip if the conjugate pad is not present;
(f) a second sample filter in operable contact with the fluid collector so that liquid passing through the second sample filter is applied to the fluid collector; and
(g) optionally, a backing in contact with one side of the chromatographic strip, the backing being situated so that fluid can pass unimpeded from the fluid-transmitting element in operable contact with the first end of the chromatographic strip and from the fluid collector or conjugate pad in operable contact with the second end of the chromatographic strip into the chromatographic strip.

61. The test strip of claim 60, wherein the fluid-transmitting element is a first sample filter.

62. A test strip for a lateral flow assay for detection of at least one analyte in a sample comprising:
(a) a chromatographic strip having a first end and a second end, the test strip including a capture band for capturing the analyte;
(b) a first sample filter in operable contact with the first end of the chromatographic strip, the first sample filter being located so that fluid applied to the first sample filter passes through the first sample filter and is applied to the chromatographic strip;
(c) at least one absorbent pad in operable contact with at least part of the first sample filter so that the at least one absorbent pad can withdraw fluid from the chromatographic strip at the first end of the chromatographic strip, the fluid being drawn back through the sample filter;
(d) optionally, a conjugate pad in operable contact with the second end of the chromatographic strip, the conjugate pad comprising a mobilizable labeled specific binding partner for the analyte;
(e) a fluid collector in operable contact with either the conjugate pad, if present, or with the second end of the chromatographic strip, if the conjugate pad is not present, so that fluid applied to the fluid collector passes through the fluid collector to the conjugate pad, if present, or to the second end of the chromatographic strip if the conjugate pad is not present;
(f) a second sample filter in operable contact with the fluid collector so that liquid passing through the second sample filter is applied to the fluid collector; and
(g) optionally, a backing in contact with one side of the chromatographic strip, the backing being situated so that fluid can pass unimpeded from the first sample filter in operable contact with the first end of the chromatographic strip and from the fluid collector or conjugate pad in operable contact with the second end of the chromatographic strip into the chromatographic strip.

63. The test strip of claim 62, wherein a mobilizable labeled specific binding partner for the analyte is located in the chromatographic strip adjacent to the second end of the chromatographic strip so that it can be mobilized by liquid passing through the fluid collector.

64. A test strip for a lateral flow assay for detection of at least one analyte in a sample comprising:
(a) a chromatographic strip comprising a first end and a second end, at least one capture band comprising an immobilized capture agent for capturing the at least one analyte, and at least one control band comprising an immobilized control agent for determination of non-specific binding;
(b) a conjugate pad, wherein the conjugate pad is in capillary contact with the second end of the chromatograph strip, and wherein the conjugate pad comprises a mobilizable detectable agent that is capable of binding to the at least one analyte or to the capture agent after capturing the analyte;
(c) a sample filter that is adjacent to the conjugate pad on the side closer to the second end, wherein the sample filter optionally comprises an agglutinating agent, and the sample filter is in capillary contact with the chromatographic strip;
(d) optionally a fluid collector that, if present, is situated between the sample filter and the chromatographic strip;
(e) optionally, a buffer pad situated at the first end of the chromatographic strip and is in capillary contact with the chromatographic strip;
(f) a first absorbent pad situated at the first end of the chromatographic strip that is in capillary contact with the chromatographic strip, either directly or indirectly; and
(g) optionally, a second absorbent pad that, if present, is in capillary contact with the first absorbent pad; wherein the test strip allows detection or quantitation of an analyte in a sample containing whole cells.

65. The test strip of claim 64 wherein the first absorbent pad is in direct capillary contact with the chromatographic strip.

66. The test strip of claim 64 wherein the first absorbent pad is in indirect capillary contact with the chromatographic strip.

67. A cassette comprising the test strip of claim 1, 15, 60, 62, or 64 wherein the cassette is adapted to be read in a device.

68. The cassette of claim 67, wherein the cassette comprises a Port-1 and a Port-2 for application of sample or buffer.

69. The cassette of claim 68, wherein Port-2 allows application of sample to the sample filter of the test strip of the cassette.

70. The cassette of claim 68, wherein Port-1 allows for application of sample to the buffer pad of the test strip of the cassette.

71. The cassette of claim 68, wherein Port-1 allows for application of buffer to the buffer pad of the test strip of the cassette.

72. A method of conducting a lateral flow assay for determination of an analyte in a sample containing a fluid comprising the steps of:
(a) providing the test strip of claim 1;
(b) applying a first aliquot of the sample onto the first membrane;
(c) allowing fluid from the first aliquot to dissolve the detectable agent;
(d) allowing the detectable agent and the fluid from the first aliquot to flow to the capture band on the chromatographic strip; and
(e) allowing analyte in the sample, if any, and the detectable agent to be captured at the capture band.

73. The method of claim 72, wherein the method further comprises the step of pre-wetting the chromatographic strip prior to application of the first sample.

74. The method of claim 73, wherein the step of pre-wetting the chromatographic strip comprises applying a buffer onto the fifth membrane.

75. The method of claim 74 wherein the buffer is selected from the group consisting of phosphate buffered saline, Ringer's solution, Hank's solution, and Tris.

76. A method of conducting a lateral flow assay for determination of an analyte in a sample containing a fluid comprising the steps of:
(a) providing the test strip of claim 15;
(b) applying a first aliquot of the sample onto the fifth membrane, wherein the fifth membrane is adjacent to the absorbent pad;
(c) applying a second aliquot of the sample onto the first membrane;
(d) allowing fluid from the second aliquot to dissolve the detectable agent;
(d) allowing the detectable agent and the fluid from the second aliquot to flow to the capture band on the chromatographic strip;
(e) allowing fluid from the second aliquot to flow to the capture band on the chromatographic strip; and
(f) allowing the analyte in the sample, if any, and the detectable agent to be captured at the capture band.

77. A method of conducting a lateral flow assay for determination of an analyte in a sample containing a fluid comprising the steps of:
(a) providing the test strip of claim 60;
(b) applying the sample onto the first membrane;
(c) allowing fluid from the first membrane to flow from the first end of the chromatographic strip towards the second end of the chromatographic strip;
(d) applying a buffer to the buffer pad;
(e) allowing the buffer to dissolve the detectable agent;
(f) allowing the detectable agent to flow to the capture band on the chromatographic strip; and
(e) allowing analyte in the sample, if any, and the detectable agent to be captured at the capture band.

78. The method of any of claim 72, 76, or 77, wherein the detectable agent is present in the conjugate pad.

79. The method of any one of claim 72, 76, or 77, wherein the detectable agent is present on the chromatographic strip.

80. The method of any one of claim 72, 76, or 77, further comprising the step of quantifying the detectable agent captured at the capture band.

81. The method of claim 80, wherein the step of quantifying the captured detectable agent at the capture band is performed by reflectance measurement.
